(19) United States
(12) Patent Application Publication
(10) Pub. No.: US 2023/0073351 A1
(43) Pub. Date: Mar. 9, 2023

(54) SELECTING BIOLOGICAL SEQUENCES FOR SCREENING TO IDENTIFY SEQUENCES THAT PERFORM A DESIRED FUNCTION

(71) Applicant: Zymergen Inc., Emeryville, CA (US)

(72) Inventors: Eyal Akiva, Foster City, CA (US); Rebecca Davidson, New York, NY (US); Stepan Tymoshenko, Emeryville, CA (US)

(73) Assignee: Zymergen Inc., Emeryville, CA (US)

(21) Appl. No.: 17/760,388

(22) PCT Filed: Feb. 12, 2021
(86) PCT No.: PCT/US2021/017913

§ 371 Date: Aug. 9, 2022

Related U.S. Application Data
(60) Provisional application No. 62/978,566, filed on Feb. 19, 2020.

(51) Int. Cl. G16B 20/00; G16B 30/00; G16B 40/20; G06N 7/00

(52) U.S. Cl. CPC G16B 20/00; G16B 30/00; G16B 40/20; G06N 7/005

(57) ABSTRACT

Systems, methods, and non-transitory computer-readable media are described for identifying candidate biological sequences for screening to determine whether the candidate biological sequences enable a biological function. Identification may be based upon (a) degrees of similarity between test sequences and reference sequences that are known to enable the function, and (b) comparisons of regions in the test sequences to reference regions that are known to bind to a molecule. Identification may also be based upon determining sequence similarities between reference sequences, grouping the reference sequences into clusters that each correspond to a molecule that is indicated as capable of being bound by a reference sequence in the cluster, and identifying matching test sequences based upon a comparison of the test sequences with reference sequences in the clusters.

### CROSS-REFERENCE TO RELATED APPLICATIONS

This application claims the benefit of priority of U.S. provisional application No. 62/978,566, filed 19 Feb. 2020. This application is related to International Application No. PCT/US19/46580, filed Aug. 14, 2019 (the “AES” application), and to U.S. application Ser. No. 15/140,296, filed on Apr. 27, 2016 (U.S. Patent Pub. No. US 2017/0316353)(the “Codon” application), which are incorporated by reference in their entirety herein.

### SEQUENCE LISTING

The instant application contains a Sequence Listing which has been submitted electronically in ASCII format and is hereby incorporated by reference in its entirety. The ASCII copy, created on Aug. 12, 2019, is named ZYM011WOPC01_SL.txt and is 39,253 bytes in size.

### FIELD

The disclosure relates generally to methods which improve genetic engineering of cells and, in particular, to efficiently identifying biological sequences (e.g., enzymes) to be screened for performance of a desired biological function.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

The critical path of designing the biomanufacturing of molecules includes the selection of proteins (e.g., enzymes, transporters, transcription factors) that perform the required functions that effectively produce a desired end product. Biologists, chemists, material scientists, and others in related disciplines employ bioengineering to produce desired molecules with desired phenotypic characteristics from cells by, for example, modifying the cell's genome. Such cells may themselves be unicellular organisms (e.g., bacteria) or components of multicellular host organisms, and may be mutated variants of cells found in nature. The process to engineer a cell to make a product of interest molecule typically requires altering the metabolism of the host cell by inserting, deleting, or regulating one or more genes that correspond to an enzymatic catalytic function of a given reaction or reactions, or that correspond to other cellular functions.

Selection of protein sequences (e.g., enzymes) that have the necessary function, or underlying DNA sequences for coding those protein sequences, from the multitude of all the known and predicted variants is often a hard-to-scale, error-prone process. A chemist or other scientist may use their knowledge and intuition to manually select the optimal enzyme candidates for catalyzing reactions along the pathways to the product of interest. However, the metabolic network of pathways can be enormous, with each pathway containing multiple reactions (e.g., 10 pathways each containing 10 reactions, or even more), for which manual determination of optimal enzymes is time-consuming and error-prone. Moreover, manual annotation of enzymes can be erroneous, and in other cases may not cause the catalyzed reaction product to be expressed to a desired degree.

Using enzymes as an example, chemical reactions catalyzed by enzymes can be carried out by proteins that are members of specific families. An enzyme family is defined as protein sequences that are similar to each other and may carry out the same or similar reaction (not necessarily upon the same substrate), as inferred from sequence or structure similarity. The main challenge of selecting enzymes that will carry out the required reaction (or proteins that will perform the required function) is that these families are frequently large and include up to hundreds of thousands of non-identical sequences. Since the capacity for experimental screening for a particular function is limited, the question is how can one adequately and cost-effectively sample the sequence diversity of a large protein family to find a manageable number of proteins sequences for functional screening?

One solution for effective sampling of large protein families is to retrieve a large set of sequences that belong to a family based on sequence similarity (e.g., by querying public sequence databases), clustering this set, and then arbitrarily selecting one sequence per cluster. This method is limited since the members of the superfamily are only predicted to carry out the same function, and a very large portion of them may be false assignments. A second limitation is that in many cases, family members are very diverse from each other, and even clustering by very low sequence identity still produces a very large set of variants that exceeds most screening capacities.

Some related art follows. Gerlt, et al., Enzyme Function Initiative-Enzyme Similarity Tool (EFI-EST): A web tool for generating protein sequence similarity networks, Biochimica et Biophysica Acta (BBA)—Proteins and Proteomics, Volume 1854, Issue 8, August 2015, Pages 1019-1037, discusses sequence similarity networks. In Akiva, et al., Evolutionary and molecular foundations of multiple contemporary functions of the nitroreductase superfamily, Proc Natl Acad Sci USA., 2017 Nov. 7, the authors use a position profiling procedure for exemplifying substrate and enzyme reaction diversity in a protein family, and describe it as a method that can be helpful for enzyme selection and design. Atkinson, et al, Using sequence similarity networks for visualization of relationships across diverse protein superfamilies, PLoS One. 2009;4(2), mentions position profiling as a method to differentiate between diverse families of specific function, and discusses applications to protein engineering and selection. Upadhyay, et al., Cache Domains That are Homologous to, but Different from PAS Domains Comprise the Largest Superfamily of Extracellular Sensors in Prokaryotes, PLoS Comput Biol. 2016 Apr. 6;12(4), discusses the assignment of a predicted function to clusters of similar sequences, and the generation of an HMM to explore the sequence space of a specific protein family in a large protein sequence database. Zallot, et al., The EFI Web Resource for Genomic Enzymology Tools: Leveraging Protein, Genome, and Metagenome Databases to Discover Novel Enzymes and Metabolic Pathways, Biochemistry 2019, 58, 41, 4169-4182 discloses generating HMMs from clusters of a sequence similarity network, running them vs. metegenomic databases and ranking the clusters by function (in order to find novel chemistries).

### SUMMARY OF THE DISCLOSURE

Embodiments of the disclosure select a manageable, limited number of diverse proteins to be screened for performance of a desired activity. This limited set is then used for functional screening for that particular activity, which is a crucial step in synthetic biology endeavors. According to embodiments of the disclosure, the input for this methodology is a protein function, and the outputs are two “bins” of sequences that sample diverse sequence variants.

Embodiments of the disclosure sample the sequence variation in an efficient manner. For example, the inventors have been able to reduce a comprehensive set of >100,000 unique enzyme sequences that belong to the same family by 60%. Each sequence in the reduced set represents a group of sequences that share very low percent sequence identity between them (e.g., 25%), but do share the same amino acids that comprise the catalytic machinery and substrate binding site of these enzymes. As in illustrative example, if 1,000 sequences share an average 25 percent seqeuence identity between them (“remote homologs”), but all of them have (or are predicted to have) the same four amino acids that catalyze the enzymatic reaction, and the same 15 amino acids that function in binding the substrate and releasing the product, they are all represented by a single sequence.

Embodiments of the disclosure provide systems, methods and non-transitory computer-readable media storing instructions for identifying candidate biological sequences for screening to determine whether the candidate biological sequences enable a biological function. Embodiments of the disclosure identify a plurality of candidate sequences in a test set of test sequences (e.g., enzymes) based at least in part upon (a) degrees of similarity between the test sequences and reference sequences, of a reference set of reference sequences, that are known to enable the function, and (b) comparisons of one or more regions in the test sequences to one or more reference regions, in one or more of the reference sequences, that are known to bind to a molecule.

The action of identifying may comprise determining a set of matching test sequences including test sequences having degrees of sequence similarity to the reference sequences that satisfy a similarity threshold; and comparing one or more regions of the matching test sequences with the one or more reference regions. Identification of the plurality of candidate sequences may employ a Hidden Markov Model (“HMM”) to determine the degrees of similarity between the test sequences and the reference sequences.

According to embodiments of the disclosure, the one or more reference regions are identified based at least in part upon an analysis of three-dimensional structures of the one or more reference sequences. According to embodiments of the disclosure, a multiple sequence alignment (“MSA”) of the reference sequences is annotated with annotations indicating the one or more reference regions, and the test sequences are added to the annotated reference MSA after aligning the test sequence to the MSA. According to embodiments of the disclosure, the comparisons of the one or more regions in the test sequences to the one or more reference regions comprises selecting a plurality of test sequences as the plurality of candidate sequences based at least in part upon the probability that a sequence component occurs at a position in the one or more reference regions.

Embodiments of the disclosure identify, in a test set of test sequences, candidate biological sequences for screening to determine whether they enable a biological function by determining sequence similarities between reference sequences, of a reference set of reference sequences, that are known to enable the biological function; grouping the reference sequences into clusters based at least in part upon their sequence similarities, wherein each cluster corresponds to a molecule that is indicated as bindable by one or more of the reference sequences in the cluster; and identifying a plurality of matching test sequences based upon a comparison of the test sequences with reference sequences in one or more of the clusters.

Embodiments of the disclosure identify matching test sequences based upon the comparison of the test sequences with reference sequences in one or more of the clusters; clustering the matching test sequences into a plurality of clusters of the matching test sequences; and selecting the candidate sequences from the plurality of clusters of the matching test sequences.

Embodiments of the disclosure employ a Hidden Markov Model (“HMM”) to determine the similarities between the test sequences and the reference sequences in the one or more clusters. According to embodiments of the disclosure, the sequence similarities between the reference sequences are statistical estimates.

According to embodiments of the disclosure, empirical performance of one or more selected candidate sequences is determined. Embodiments of the disclosure add one or more first candidate sequences to the reference set based at least in part upon empirical performance of the one or more first candidate sequences.

Embodiments of the disclosure produce a desired molecule employing one or more candidate biological sequences that enable one or more functions used to produce the desired molecule, wherein the one or more candidate biological sequences are identified using the approaches described above. According to embodiments of the disclosure, the one or more candidate biological sequences are enzymes that catalyze at least one reaction pathway leading to the desired molecule.

### BRIEF DESCRIPTION OF THE DRAWINGS

FIG. 1A is a system diagram of a laboratory information management system (LIMS) of embodiments of the disclosure for the high-throughput (“HTP”) design, building, testing, and analysis of DNA sequences.

FIG. 1B illustrates a distributed system of embodiments of the disclosure.

FIG. 1C and FIG. 1D are corresponding flow diagrams for the LIMS.

FIGS. 2A and 2B depict steps for DNA assembly, transformation, and strain screening, according to embodiments of the disclosure.

FIGS. 3A and 3B provide another view of high-throughput strain engineering, according to embodiments of the disclosure.

FIG. 4 illustrates an automated system of embodiments of the disclosure.

FIG. 5 illustrates the operation of algorithmic biological sequence selection according to embodiments of the disclosure.

FIGS. 6A-6H illustrate an example of identifying at least one sequence to enable tyrosine decarboxylase activity, according to embodiments of the disclosure. FIG. 6A discloses SEQ ID NOS 1-6, respectively, in order of appearance. FIG. 6B discloses SEQ ID NOS 7-10, respectively, in order of appearance.

FIG. 7 illustrates two different approaches for selecting biological sequences for screening to determine whether they perform a desired biological function, according to embodiments of the disclosure.

FIG. 8 illustrates alignment of a test sequence to a multiple sequence alignment of reference sequences that are annotated according to strategic regions, according to embodiments of the disclosure.

FIG. 9 illustrates a cloud computing environment according to embodiments of the disclosure.

FIG. 10 illustrates an example of a computer system that may be used to execute instructions stored in a non-transitory computer readable medium (e.g., memory) in accordance with embodiments of the disclosure.

### DETAILED DESCRIPTION

The present description is made with reference to the accompanying drawings, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

Glossary

A biological sequence may be a sequence of nucleotides or amino acids.

To clarify, unless otherwise indicated herein, the term “molecule” refers to a type of molecule (e.g., a particular type of protein molecule), and not to an individual isolated molecule.

Similarly, to clarify, unless otherwise indicated herein, the term “cell” refers to a type of cell, and not to an individual isolated cell.

The terms “organism” “microorganism” or “microbe” are used interchangeably herein and include the two prokaryotic domains, Bacteria and Archaea, as well as certain eukaryotic fungi, yeasts, and protists.

A “high-throughput (HTP)” method of genomic engineering may involve the utilization of at least one piece of automated equipment (e.g., a liquid handler or plate handler machine) to carry out at least one step of embodiments of the disclosure.

Genomic Automation

Automation of the methods of the disclosure enables high-throughput phenotypic screening and identification of target products from multiple test strain variants simultaneously. Hundreds or thousands of mutant strains may be constructed in a high-throughput fashion. The robotic and computer systems described below are the structural mechanisms by which such a high-throughput process can be carried out.

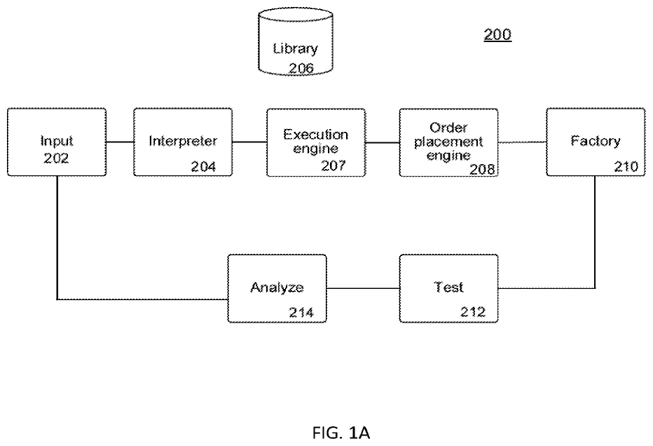

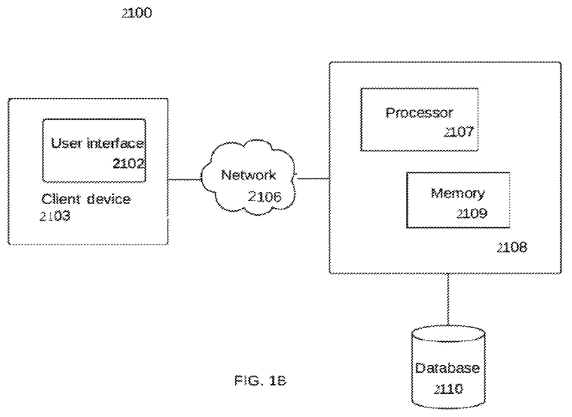

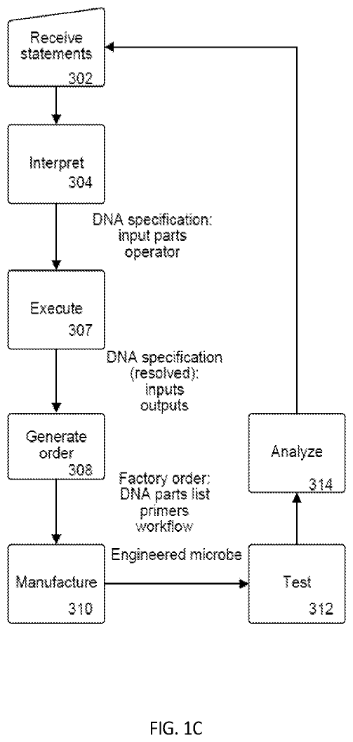

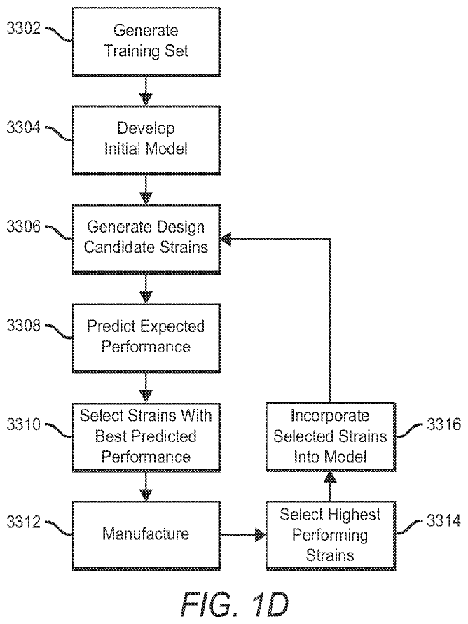

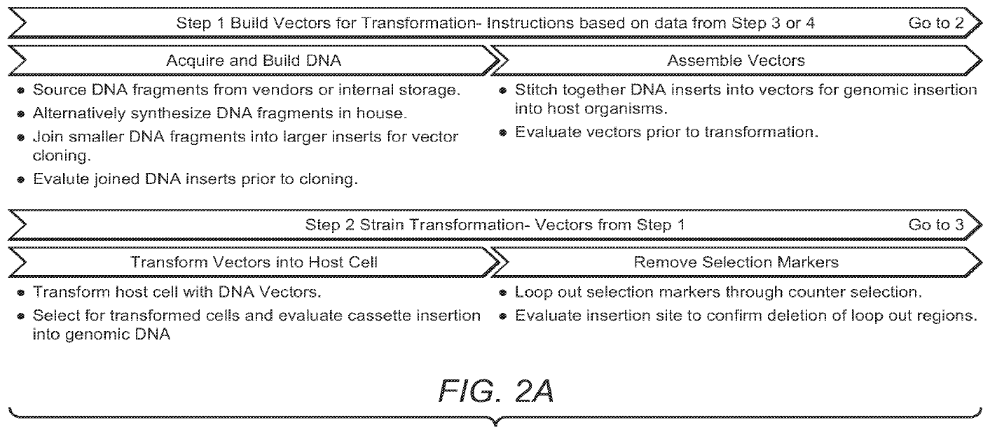

FIG. 1A is a system diagram of a laboratory information management system (LIMS) 200 of embodiments of the disclosure for the high-throughput (“HTP”) design, building, testing, and analysis of DNA sequences.

FIG. 1B illustrates a distributed system 2100 of embodiments of the disclosure. A user interface 2102 includes a client-side interface such as a text editor or a graphical user interface (GUI). The user interface 2102 may reside at a client-side computing device 2103, such as a laptop or desktop computer. The client-side computing device 2103 is coupled to one or more servers 2108 through a network 2106, such as the Internet.

The server(s) 2108 are coupled locally or remotely to one or more databases 2110, which may include one or more corpora of libraries including data such as genome data, genetic modification data (e.g., promoter ladders), process condition data, strain environmental data, and phenotypic performance data that may represent microbial strain performance at both small and large scales, and in response to genetic modifications.

In embodiments, the server(s) 2108 include at least one processor 2107 and at least one memory 109 storing instructions that, when executed by the processor(s) 2107, perform operations disclosed herein, including, e.g., annotating biological sequences, predicting performance of biological sequences, developing statistical models of biological sequences, comparing biological sequences and models, and selecting biological sequences for screening. The same arrangement may also act as the analysis equipment 214 or other elements of the LIMS system involved in the design, manufacture, test, and analysis of microbial strains, according to embodiments of the disclosure. A computing system, such as that of a server 2108 or local computer, that performs any of these operations may be referred to as an “engine” herein. In some instances herein, a particular type of engine (e.g., execution engine 207) is specified. In other instances the type of “engine” is not particularly specified, in which cases the term “engine” shall be understood to describe a computing system such as that discussed above that performs operations described as associated with such an engine.

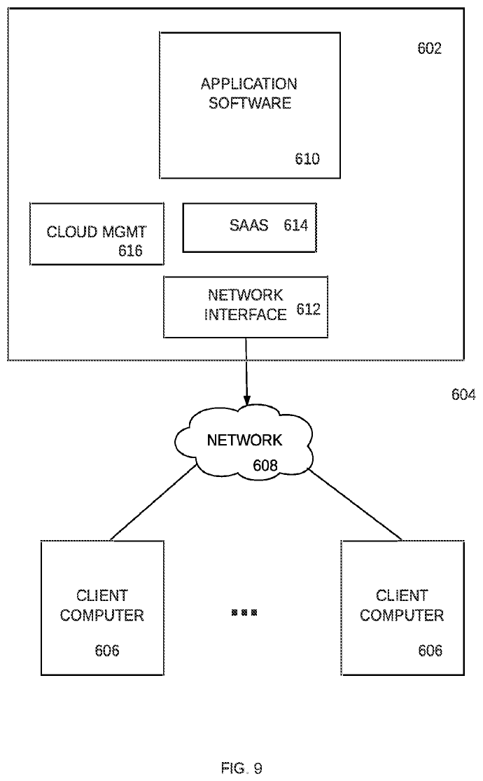

Alternatively, the software and associated hardware for the engine may reside locally at the client 2103 instead of at the server(s) 2108, or be distributed between both client 2103 and server(s) 2108. In embodiments, all or parts of the engine may run as a cloud-based service, depicted further in FIG. 9.

The database(s) 2110 may include public databases, as well as custom databases generated by the user or others, e.g., databases including molecules generated via fermentation experiments performed by the user or third-party contributors. The database(s) 2110 may be local or remote with respect to the client 2103 or distributed both locally and remotely.

FIG. 1C and FIG. 1D are corresponding flow diagrams for LIMS 200. In embodiments of LIMS, many changes may be made to an input DNA sequence at a time, resulting in a single output sequence for each change or change set. To optimize strains (e.g., manufacture microbes that efficiently produce an organic compound with high yield), LIMS may produce many such DNA output sequences at a time, so that they may be analyzed within the same timeframe to determine which host cells, and thus which modifications to the input sequence, best achieve the desired properties.

In some embodiments the system enables the design of multiple nucleotide sequence constructs (such as DNA constructs like promoters, codons, or genes), each with one or more changes, and creates a work order (i.e., “factory order”) to instruct a gene manufacturing system, factory 210, to build the nucleotide sequence constructs in the form of microbes carrying the constructs. Examples of microbes that may be built include, without limitation, hosts such as bacteria, fungi, and yeast. According to the system, the microbes are then tested for their properties (e.g., yield, titer). In feedback-loop fashion, the results are analyzed to iteratively improve upon the designs of prior generations to achieve more optimal microbe performance.

Although the design, build, test and analysis process is described herein primarily in the context of microbial genome modification, those skilled in the art will recognize that this process may be used for desired gene modification and expression goals in any type of host cell.

Referring to FIGS. 1A-1D in more detail, an input interface 1202, such as a computer running a program editor, receives statements of a program/script that is used to design one or more DNA output sequences (see 302). Such a genomic design program language may be referred to herein as the “Codon” programming language developed by the assignee of the present disclosure, and described herein in the Codon application referenced above. A powerful feature of embodiments of the disclosure is the ability to develop designs for a very large number of DNA sequences (e.g., microbial strains, plasmids) within the same program with just a few procedural statements.

Here, the editor enables a user to enter and edit the program, e.g., through graphical or text entry or via menus or forms using a keyboard and mouse on a computing device. Those skilled in the art will recognize that other input interfaces 202 may be employed without the need for direct user input, e.g., the input interface 202 may employ an application programming interface (API), and receive statements in files comprising the program from another computing device. The input interface 202 may communicate with other elements of the system over local or remote connections.

As described in the Codon application, an interpreter or compiler/execution unit 204 evaluates program statements into novel DNA specification data structures of embodiments of the disclosure (304). According to embodiments of the disclosure, the interpreter 204, along with the execution engine 207 and the order placement engine 208 transforms the program statements from a logical specification into a specification of a physical manufacturing process for use by the factory 210.

The factory order placer 208 can determine the intermediate parts that will be required for that workflow process performed by the factory 210 using libraries of known parameters and known algorithms that obey known heuristics and other properties (e.g., optimal melting temperature to run on common equipment).

The resulting factory order may include a combination of a prescribed set of steps, as well as the parameters, inputs and outputs for each of those steps for each DNA sequence to be constructed. The factory order may include a DNA parts list including a starting microbial base strain, a list of primers, guide RNA sequences, or other template components or reagent specifications necessary to effect the workflow, along with one or more manufacturing workflow specifications for different operations within the DNA specification. These primary, intermediate, and final parts or strains may be reified via a factory build graph; the workflow steps refer to elements of the build graph with various roles. The order placement engine 208 may refer to the library 206 for the information discussed above. According to embodiments of the disclosure, this information is used to reify the design campaign operations in physical (as opposed to in silico) form at the factory 210 based upon conventional techniques for nucleotide sequence synthesis, as well as custom techniques developed by users or others.

For example, assume a recursive program statement has a top-level function of circularize and its input is a chain of concatenate specifications. The factory order placer 208 may interpret that series of inputs such that a person or robot in the lab may perform a PCR reaction to amplify each of the inputs and then assemble them into a circular plasmid, according to conventional techniques or custom/improved techniques developed by the user. The factory order may specify the PCR products that should be created in order to do the assembly. The factory order may also provide the primers that should be purchased in order to perform the PCR.

In another example, assume a program statement specifies a top-level function of replace. The factory order placer 208 may interpret this as a cell transformation (a process that replaces one section of a genome with another in a live cell). Furthermore, the inputs to the replace function may include parameters that indicate the source of the DNA (e.g. cut out of another plasmid, amplified off some other strain).

The order placement engine 208 may communicate the factory order to the factory 210 over local or remote connections. Based upon the factory order, the factory 210 may acquire short DNA parts from outside vendors and internal storage, and employ techniques known in the art, such as the Gibson assembly protocol or the Golden Gate Assembly protocol, to assemble DNA sequences corresponding to the input designs (310). The factory order itself may specify which techniques to employ during beginning, intermediate and final stages of manufacture. For example, many laboratory protocols include a PCR amplification step that requires a template sequence and two primer sequences. The factory 210 may be implemented partially or wholly using robotic automation.

According to embodiments of the disclosure, the factory order may specify the production in the factory 210 of hundreds or thousands of DNA constructs, each with a different genetic makeup. The DNA constructs are typically circularized to form plasmids for insertion into the base strain. In the factory 210, the base strain is prepared to receive the assembled plasmid, which is then inserted.

The resulting DNA sequences assembled at the factory 210 are tested using test equipment 212 (312). During testing, the microbe strains are subjected to quality control (QC) assessments based upon size and sequencing methods. The resulting, modified strains that pass QC may then be transferred from liquid or colony cultures on to plates. Under environmental conditions that model production conditions, the strains are grown and then assayed to test performance (e.g., desired product concentration). The same test process may be performed in flasks or tanks.

In feedback-loop fashion, the results may be analyzed by analysis equipment 214 to determine which microbes exhibit desired phenotypic properties (314). During the analysis phase, the modified strain cultures are evaluated to determine their performance, i.e., their expression of desired phenotypic properties, including the ability to be produced at industrial scale. The analysis phase uses, among other things, image data of plates to measure microbial colony growth as an indicator of colony health. The analysis equipment 214 may include a computer to perform a number of operations described herein, including correlating genetic changes with phenotypic performance, and saving the resulting genotype-phenotype correlation data in libraries, which may be stored in library 206, to inform future microbial production.

LIMS iterates the design/build/test/analyze cycle based on the correlations developed from previous factory runs. During a subsequent cycle, the analysis equipment 214, alone or in conjunction with human operators, may select the best candidates as base strains for input back into input interface 202, using the correlation data to fine tune genetic modifications to achieve better phenotypic performance with finer granularity. In this manner, the laboratory information management system of embodiments of the disclosure implements a quality improvement feedback loop.

Those skilled in the art will recognize that some embodiments described herein may be performed entirely through automated means of the LIMS system 200, e.g., by the analysis equipment 214, or by human implementation, or through a combination of automated and manual means. When an operation is not fully automated, the elements of the LIMS system 200, e.g., analysis equipment 214, may, for example, receive the results of the human performance of the operations rather than generate results through its own operational capabilities. As described elsewhere herein, components of the LIMS system 200, such as the analysis equipment 214, may be implemented wholly or partially by one or more computer systems. In some embodiments, in particular where operations are performed by a combination of automated and manual means, the analysis equipment 214 may include not only computer hardware, software or firmware (or a combination thereof), but also equipment operated by a human operator such as that listed in Table 1 below.

In some embodiments, the high-throughput screening process is designed to predict performance of strains in bioreactors. Culture conditions may be selected to be suitable for the organism and reflective of bioreactor conditions. Individual colonies may be picked and transferred into 96 well plates and incubated for a suitable amount of time. Cells may be subsequently transferred to new 96 well plates for additional seed cultures, or to production cultures. Cultures may be incubated for varying lengths of time, where multiple measurements may be made. These may include measurements of product, biomass or other characteristics that predict performance of strains in bioreactors. High-throughput culture results may be used to predict bioreactor performance.

In some embodiments, tank-based performance validation is used to confirm performance of strains isolated by high throughput screening. Fermentation processes/conditions may be obtained from customers of the operator of the LIMS system. Candidate strains may be screened using bench scale fermentation reactors (e.g., reactors disclosed in Table 1 of the present disclosure) for relevant strain performance characteristics such as productivity or yield.

Iterative Strain Design Optimization

Referring to FIGS. 1A-1C, the order placement engine 208 places a factory order to the factory 210 to manufacture microbial strains incorporating the candidate mutations, according to embodiments of the disclosure. In feedback-loop fashion, the results may be analyzed by the analysis equipment 214 to determine which microbes exhibit desired phenotypic properties (314). During the analysis phase, the modified strain cultures are evaluated to determine their performance, e.g., their expression of desired phenotypic properties, including, e.g., the ability to be produced at industrial scale. For example, the analysis phase uses, among other things, image data of plates to measure microbial colony growth as an indicator of colony health. The analysis equipment 214 may be used to correlate genetic changes with phenotypic performance, and save the resulting genotype-phenotype correlation data in libraries, which may be stored in library 206, to inform future microbial production.

In particular, the genotype-phenotype correlation data resulting from candidate changes that result in sufficiently high measured performance may be added to a training data set. In this manner, the best performing mutations are added to a predictive strain design model in a supervised machine learning fashion.

According to embodiments of the disclosure, LIMS iterates the design/build/test/analyze cycle based on the correlations developed from previous factory runs. During a subsequent cycle, the analysis equipment 214 alone, or in conjunction with human operators, may select the best candidates as base strains for input back into input interface 202, using the correlation data to fine tune genetic modifications to achieve better phenotypic performance with finer granularity. In this manner, the laboratory information management system of embodiments of the disclosure implements a quality improvement feedback loop.

In sum, with reference to the flowchart of FIG. 1D the iterative predictive strain design workflow of embodiments of the disclosure may be described as follows:
Generate a training set of input and output variables, e.g., genetic changes as inputs and performance features as outputs (3302). Generation may be performed by the analysis equipment 214 based upon previous genetic changes and the corresponding measured performance of the microbial strains incorporating those genetic changes.
Develop an initial model (e.g., linear regression model) based upon training set (3304). This may be performed by the analysis equipment 214.
Generate design candidate strains (3306)
   In one embodiment, the analysis equipment 214 may fix the number of genetic changes to be made to a background strain, in the form of combinations of changes. To represent these changes, the analysis equipment 214 may provide to the interpreter 204 one or more DNA specification expressions representing those combinations of changes. (These genetic changes or the microbial strains incorporating those changes may be referred to as “test inputs.”) The interpreter 204 interprets the one or more DNA specifications, and the execution engine 207 executes the DNA specifications to populate the DNA specification with resolved outputs representing the individual candidate design strains for those changes.
Based upon the model, the analysis equipment 214 predicts expected performance of each candidate design strain (3308).
The analysis equipment 214 selects a limited number of candidate designs, e.g., 100, with highest predicted performance (3310).
   The analysis equipment 214 may account for second-order effects such as epistasis, by, e.g., filtering top designs for epistatic effects, or factoring epistasis into the predictive model.
Build the filtered candidate strains (at the factory 210) based on the factory order generated by the order placement engine 208 (3312).
The analysis equipment 214 measures the actual performance of the selected strains, selects a limited number of those selected strains based upon their superior actual performance (3314), and adds the design changes and their resulting performance to the predictive model (3316). The predictive model may employ linear regression.
The analysis equipment 214 then iterates back to generation of new design candidate strains (3306), and continues iterating until a stop condition is satisfied. The stop condition may comprise, for example, the measured performance of at least one microbial strain satisfying a performance metric, such as yield, growth rate, or titer.

In the example above, the iterative optimization of strain design may employ feedback and linear regression to implement machine learning.

Other General HTP Descriptions

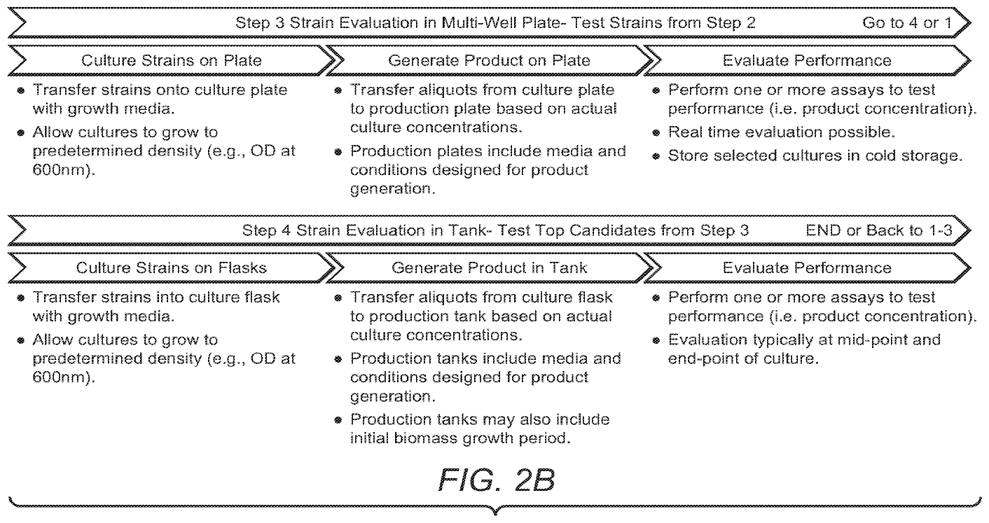

FIGS. 2A and 2B depict steps for DNA assembly, transformation, and strain screening, according to embodiments of the disclosure. FIG. 2A depicts steps for building DNA fragments, cloning DNA fragments into vectors, transforming the vectors into host strains, and removing selection markers. FIG. 2B depicts steps for high-throughput culturing, screening, and evaluation of selected host strains. This figure also depicts optional steps of culturing, screening, and evaluating selected strains in culture tanks.

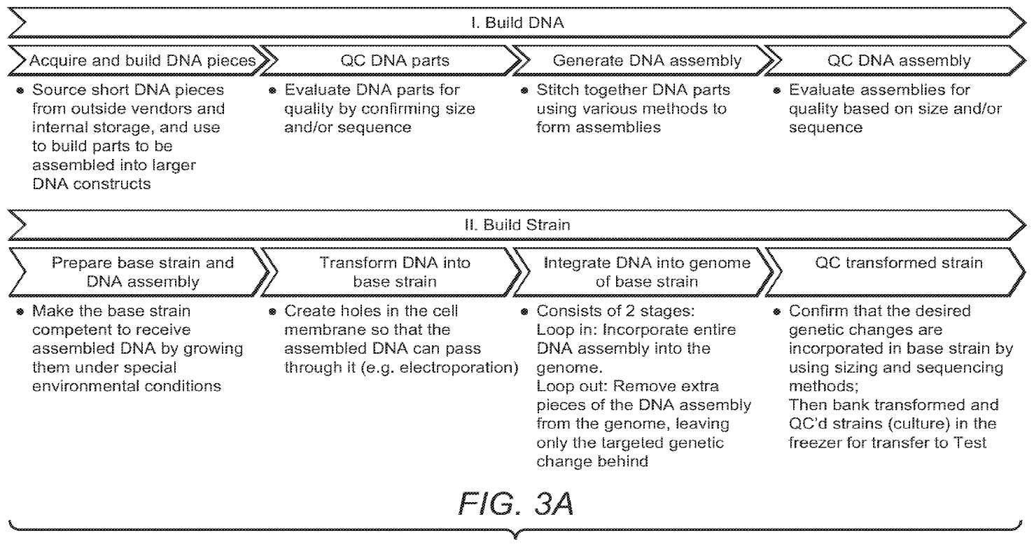

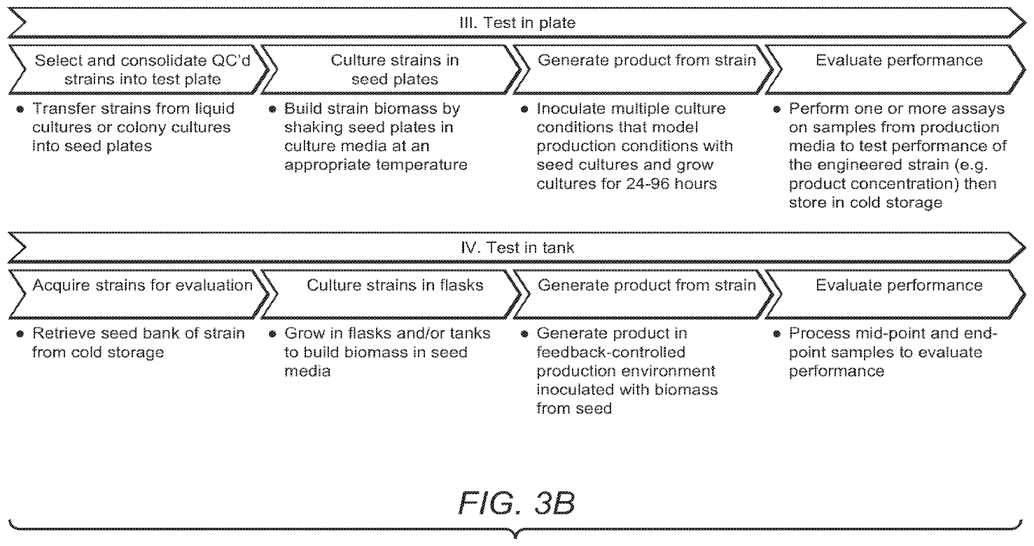

FIGS. 3A and 3B provide another view of high-throughput strain engineering, according to embodiments of the disclosure. The flow chart depicts steps for building DNA, building strains from the DNA, and testing strains in plates and in tanks.

HTP Robotic Systems

According to embodiments of the disclosure, the automated HTP methods of the disclosure comprise a robotic system. The systems outlined herein are generally directed to the use of 96- or 384-well microtiter plates, but as will be appreciated by those in the art, any number of different plates or configurations may be used. In addition, any or all of the steps outlined herein may be completely or partially automated.

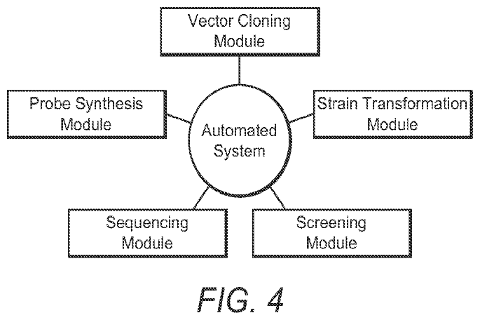

Referring to FIG. 4, automated systems of embodiments of the disclosure comprise one or more work modules. For example, in some embodiments, automated robotic systems system include a DNA synthesis module, a vector cloning module, a strain transformation module, a screening module, and a sequencing module capable of cloning, transforming, culturing, screening and sequencing host organisms.

As will be appreciated by those in the art, an automated system can include a wide variety of components, including, but not limited to: liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and computer systems.

In some embodiments, the robotic systems of the present disclosure include automated liquid and particle handling enabling high-throughput pipetting to perform all the steps in the process of gene targeting and recombination applications. This includes liquid and particle manipulations such as aspiration, dispensing, mixing, diluting, washing, accurate volumetric transfers; retrieving and discarding of pipette tips; and repetitive pipetting of identical volumes for multiple deliveries from a single sample aspiration. These manipulations are cross-contamination-free liquid, particle, cell, and organism transfers. The instruments perform automated replication of microplate samples to filters, membranes, and/or daughter plates, high-density transfers, full-plate serial dilutions, and high capacity operation.

In some embodiments, the customized automated liquid handling system of the disclosure is a TECAN machine (e.g. a customized TECAN Freedom Evo).

In some embodiments, the automated systems of the present disclosure are compatible with platforms for multi-well plates, deep-well plates, square well plates, reagent troughs, test tubes, mini tubes, microfuge tubes, cryovials, filters, micro array chips, optic fibers, beads, agarose and acrylamide gels, and other solid-phase matrices or platforms are accommodated on an upgradeable modular deck. In some embodiments, the automated systems of the present disclosure contain at least one modular deck for multi-position work surfaces for placing source and output samples, reagents, sample and reagent dilution, assay plates, sample and reagent reservoirs, pipette tips, and an active tip-washing station.

In some embodiments, the automated systems of the present disclosure include high-throughput electroporation systems. In some embodiments, the high-throughput electroporation systems are capable of transforming cells in 96 or 384- well plates. In some embodiments, the high-throughput electroporation systems include VWR® High-throughput Electroporation Systems, BTX™, Bio-Rad® Gene Pulser MXcell™ or other multi-well electroporation system.

In some embodiments, the integrated thermal cycler and/or thermal regulators are used for stabilizing the temperature of heat exchangers such as controlled blocks or platforms to provide accurate temperature control of incubating samples from 0° C. to 100° C.

In some embodiments, the automated systems of the present disclosure are compatible with interchangeable machine-heads (single or multi-channel) with single or multiple magnetic probes, affinity probes, replicators or pipetters, capable of robotically manipulating liquid, particles, cells, and multi-cellular organisms. Multi-well or multi-tube magnetic separators and filtration stations manipulate liquid, particles, cells, and organisms in single or multiple sample formats.

In some embodiments, the automated systems of the present disclosure are compatible with camera vision and/or spectrometer systems. Thus, in some embodiments, the automated systems of the present disclosure are capable of detecting and logging color and absorption changes in ongoing cellular cultures.

In some embodiments, the automated system of the present disclosure is designed to be flexible and adaptable with multiple hardware add-ons to allow the system to carry out multiple applications. The software program modules allow creation, modification, and running of methods. The system's diagnostic modules allow setup, instrument alignment, and motor operations. The customized tools, labware, and liquid and particle transfer patterns allow different applications to be programmed and performed. The database allows method and parameter storage. Robotic and computer interfaces allow communication between instruments.

Persons having skill in the art will recognize the various robotic platforms capable of carrying out the HTP engineering methods of the present disclosure. Table 1 below provides a non-exclusive list of scientific equipment capable of carrying out each step of the HTP engineering steps of the present disclosure, such as those described in FIGS. 3A-3B.

TABLE 1
Non-exclusive list of Scientific Equipment Compatible with the HTP engineering methods of the disclosure

| | | | Compatible Equipment |
|---|---|---|---|
| | Equipment Type | Operation(s) performed | Make/Model/Configuration |
| Acquire and | liquid handlers | Hitpicking (combining by | Hamilton Microlab STAR, Labcyte |
| build DNA | | transferring) | Echo 550, Tecan EVO 200, Beckman |
| pieces | | primers/templates for PCR | Coulter Biomek FX, or equivalents |
| | | amplification of DNA parts | |
| | Thermal cyclers | PCR amplification of DNA | Inheco Cycler, ABI 2720, ABI |
| | | parts | Proflex 384, ABI Veriti, or |
| | | | equivalents |
| QC DNA | Fragment analyzers | gel electrophoresis to | Agilent Bioanalyzer, AATI |
| parts | (capillary | confirm PCR products of | Fragment Analyzer, or equivalents |
| | electrophoresis) | appropriate size | |
| | Sequencer | Verifying sequence of | Beckman Ceq-8000, Beckman |
| | (sanger: Beckman) | parts/templates | GenomeLab ™, or equivalents |
| | NGS (next generation | Verifying sequence of | Illumina MiSeq series sequences, |
| | sequencing) instrument | parts/templates | illumina Hi-Seq, Ion torrent, pac |
| | | | bio or other equivalents |
| | nanodrop/plate | assessing concentration of | Molecular Devices SpectraMax |
| | reader | DNA samples | M5, Tecan M1000, or equivalents. |
| Generate DNA | liquid handlers | Hitpicking (combining by | Hamilton Microlab STAR, Labcyte |
| assembly | | transferring) DNA parts | Echo 550, Tecan EVO 200, Beckman |
| | | for assembly along with | Coulter Biomek FX, or equivalents |
| | | cloning vector, addition of | |
| | | reagents for assembly | |
| | | reaction/process | |
| QC DNA | Colony pickers | for inoculating colonies in | Scirobotics Pickolo, Molecular |
| assembly | | liquid media | Devices QPix 420 |
| | liquid handlers | Hitpicking primers/templates, | Hamilton Microlab STAR, Labcyte |
| | | diluting samples | Echo 550, Tecan EVO 200, Beckman |
| | | | Coulter Biomek FX, or equivalents |
| | Fragment analyzers | gel electrophoresis to | Agilent Bioanalyzer, AATI |
| | (capillary | confirm assembled products | Fragment Analyzer |
| | electrophoresis) | of appropriate size | |
| | Sequencer | Verifying sequence of | ABI3730 Thermo Fisher, Beckman |
| | (sanger: Beckman) | assembled plasmids | Ceq-8000, Beckman GenomeLab ™, |
| | | | or equivalents |
| | NGS (next generation | Verifying sequence of | Illumina MiSeq series sequences, |
| | sequencing) instrument | assembled plasmids | illumina Hi-Seq, Ion torrent, pac |
| | | | bio or other equivalents |
| Prepare base | centrifuge | spinning/pelleting cells | Beckman Avanti floor centrifuge, |
| strain and DNA | | | Hettich Centrifuge |
| assembly | | | |
| Transform DNA | Electroporators | electroporative | BTX Gemini X2, BIO-RAD |
| into base strain | | transformation of cells | MicroPulser Electroporator |
| | Ballistic | ballistic transformation of | BIO-RAD PDS1000 |
| | transformation | cells | |
| | Incubators, | for chemical | Inheco Cycler, ABI 2720, ABI |
| | thermal cyclers | transformation/heat shock | Proflex 384, ABI Veriti, or |
| | | | equivalents |
| | Liquid handlers | for combining DNA, cells, | Hamilton Microlab STAR, Labcyte |
| | | buffer | Echo 550, Tecan EVO 200, Beckman |
| | | | Coulter Biomek FX, or equivalents |
| Integrate DNA | Colony pickers | for inoculating colonies in | Scirobotics Pickolo, Molecular |
| into genome of | | liquid media | Devices QPix 420 |
| base strain | Liquid handlers | For transferring cells onto | Hamilton Microlab STAR, Labcyte |
| | | Agar, transferring from | Echo 550, Tecan EVO 200, Beckman |
| | | culture plates to different | Coulter Biomek FX, or equivalents |
| | | culture plates (inoculation | |
| | | into other selective media) | |
| | Platform | incubation with shaking of | Kuhner Shaker ISF4-X, Infors-ht |
| | shaker-incubators | microtiter plate cultures | Multitron Pro |
| QC transformed | Colony pickers | for inoculating colonies in | Scirobotics Pickolo, Molecular |
| strain | | liquid media | Devices QPix 420 |
| | liquid handlers | Hitpicking primers/templates, | Hamilton Microlab STAR, Labcyte |
| | | diluting samples | Echo 550, Tecan EVO 200, Beckman |
| | | | Coulter Biomek FX, or equivalents |
| | Thermal cyclers | cPCR verification of strains | Inheco Cycler, ABI 2720, ABI |
| | | | Proflex 384, ABI Veriti, or equivalents |
| | Fragment analyzers | gel electrophoresis to | Infors-ht Multitron Pro, Kuhner |
| | (capillary | confirm cPCR products of | Shaker ISF4-X |
| | electrophoresis) | appropriate size | |
| | Sequencer | Sequence verification of | Beckman Ceq-8000, Beckman |
| | (sanger: Beckman) | introduced modification | GenomeLab ™, or equivalents |
| | NGS (next generation | Sequence verification of | Illumina MiSeq series sequences, |
| | sequencing) instrument | introduced modification | illumina Hi-Seq, Ion torrent, pac bio |
| | | | or other equivalents |
| Select and | Liquid handlers | For transferring from | Hamilton Microlab STAR, Labcyte |
| consolidate QC’ | | culture plates to different | Echo 550, Tecan EVO 200, Beckman |
| d strains into | | culture plates (inoculation | Coulter Biomek FX, or equivalents |
| test plate | | into production media) | |
| | Colony pickers | for inoculating colonies in | Scirobotics Pickolo, Molecular |
| | | liquid media | Devices QPix 420 |
| | Platform | incubation with shaking of | Kuhner Shaker ISF4-X, Infors-ht |
| | shaker-incubators | microtiter plate cultures | Multitron Pro |
| Culture strains | Liquid handlers | For transferring from | Hamilton Microlab STAR, Labcyte |
| in seed plates | | culture plates to different | Echo 550, Tecan EVO 200, Beckman |
| | | culture plates (inoculation | Coulter Biomek FX, or equivalents |
| | | into production media) | |
| | Platform | incubation with shaking of | Kuhner Shaker ISF4-X, Infors-ht |
| | shaker-incubators | microtiter plate cultures | Multitron Pro |
| | liquid dispensers | Dispense liquid culture media | Well mate (Thermo), Benchcel2R |
| | | into microtiter plates | (velocity 11), plateloc (velocity 11) |
| | microplate labeler | apply barcoders to plates | Microplate labeler (a2+ cab − agilent), |
| | | | benchcell 6R (velocity 11) |
| Generate product | Liquid handlers | For transferring from | Hamilton Microlab STAR, Labcyte |
| from strain | | culture plates to different | Echo 550, Tecan EVO 200, Beckman |
| | | culture plates (inoculation | Coulter Biomek FX, or equivalents |
| | | into production media) | |
| | Platform | incubation with shaking of | Kuhner Shaker ISF4-X, Infors-ht |
| | shaker-incubators | microtiter plate cultures | Multitron Pro |
| | liquid dispensers | Dispense liquid culture media | well mate (Thermo), Benchcel2R |
| | | into multiple microtiter plates | (velocity 11), plateloc (velocity 11) |
| | | and seal plates | |
| | microplate labeler | Apply barcodes to plates | microplate labeler (a2+ cab − agilent), |
| | | | benchcell 6R (velocity 11) |
| Evaluate | Liquid handlers | For processing culture broth | Hamilton Microlab STAR, Labcyte |
| performance | | for downstream analytical | Echo 550, Tecan EVO 200, Beckman |
| | | | Coulter Biomek FX, or equivalents |
| | UHPLC, HPLC | quantitative analysis of | Agilent 1290 Series UHPLC and 1200 |
| | | precursor and target | Series HPLC with UV and RI detectors, |
| | | compounds | or equivalent; also any LC/MS |
| | LC/MS | highly specific analysis of | Agilent 6490 QQQ and 6550 QTOF |
| | | precursor and target | coupled to 1290 Series UHPLC |
| | | compounds as well as side | |
| | | and degradation products | |
| | Spectrophotometer | Quantification of different | Tecan M1000, spectramax M5, |
| | | compounds using | Genesys 10S |
| | | spectrophotometer based | |
| | | assays | |
| Culture strains | Fermenters: | incubation with shaking | Sartorius, DASGIPs (Eppendorf), |
| in flasks | | | BIO-FLOs (Sartorius-stedim). Applikon |
| | Platform shakers | | innova 4900, or any equivalent |
| Generate product | Fermenters: DASGIPs (Eppendorf), BIO-FLOs (Sartorius-stedim) | | |
| from strain | | | |
| Evaluate | Liquid handlers | For transferring from culture | Hamilton Microlab STAR, Labcyte |
| performance | | plates to different culture | Echo 550, Tecan EVO 200, Beckman |
| | | plates (inoculation into | Coulter Biomek FX, or equivalents |
| | | production media) | |
| | UHPLC, HPLC | quantitative analysis of | Agilent 1290 Series UHPLC and 1200 |
| | | precursor and target | Series HPLC with UV and RI detectors, |
| | | compounds | or equivalent; also any LC/MS |
| | LC/MS | highly specific analysis of | Agilent 6490 QQQ and 6550 QTOF |
| | | precursor and target | coupled to 1290 Series UHPLC |
| | | compounds as well as side | |
| | | and degradation products | |
| | Flow cytometer | Characterize strain | BD Accuri, Millipore Guava |
| | | performance (measure | |
| | | viability) | |
| | Spectrophotometer | Characterize strain | Tecan M1000, Spectramax M5, or other |
| | | performance (measure | equivalents |
| | | biomass) | |

Genome engineering. Embodiments of the disclosure identify biological sequences that enable functions in a host cell, and enable the host cell to use an identified biological sequence (e.g., by engineering the sequence into the host cell genome) to produce molecules of a desired product. Based upon their choice of target molecules, the user may instruct the tool to provide, to a gene manufacturing system, indications of the genetic sequences for the enzymes or other catalysts used to catalyze the reactions in the reaction pathways leading to each selected target molecule. The gene manufacturing system may then embody (through, e.g., insertion, replacement, deletion) the indicated genetic sequences into the genome of the host, to thereby produce an engineered genome for manufacture of the viable target molecules. In embodiments, the gene manufacturing system may be implemented using systems and techniques known in the art, or using the factory 210 described in pending U.S. patent application Ser. No. 15/140,296, filed Apr. 27, 2016, published Nov. 2, 2017, entitled “Microbial Strain Design System and Methods for Improved Large Scale Production of Engineered Nucleotide Sequences,” incorporated by reference in its entirety herein. As described in that application, the gene manufacturing system may employ known techniques such as the Gibson and Golden Gate assembly protocols to assemble DNA sequences based upon input designs. The DNA constructs are typically circularized to form plasmids for insertion into a base strain. In the gene manufacturing system, the base strain is prepared to receive the assembled plasmid, which is then inserted. Input information may include techniques to employ during beginning, intermediate and final stages of manufacture. For example, many laboratory protocols include a PCR amplification step that requires a template sequence and two primer sequences. As is known in the art, the gene manufacturing system may be implemented partially or wholly using robotic automation. In embodiments, in addition to or as a substitute for embodying genetic sequences into the host, the engine provides to the factory an indication of one or more catalysts for the factory to introduce the one or more catalysts into the growth medium of the host cell for production of the target molecule.

Production of product of interest. Embodiments of the disclosure use well-known techniques to produce a target molecule from a base strain having a native or engineered genome. According to embodiments of the disclosure, the organism is transferred to a bioreactor containing feedstock for fermentation. Under controlled conditions, the organism ferments to produce a desired product of interest (e.g., small molecule, peptide, synthetic compound, fuel, alcohol) based upon the assembled DNA.

Different types of microbes can function as platform organisms in industrial biotechnology, including bacteria and yeasts fermenting sugar compounds into end-products, as well as microalgae via photosynthesis (phototrophic algae) or fermentation (heterotrophic algae).

The bacteria or other cells can be cultured in conventional nutrient media modified as appropriate for desired biosynthetic reactions or selections. Culture conditions, such as temperature, pH and the like, are those suitable for use with the host cell selected for expression, and will be apparent to those skilled in the art. Many references are available for the culture and production of cells, including cells of bacterial, plant, animal (including mammalian) and archaebacterial origin. See e.g., Sambrook, Ausubel (all supra), as well as Berger, Guide to Molecular Cloning Techniques, Methods in Enzymology volume 152 Academic Press, Inc., San Diego, Calif.; and Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelle et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue from Sigma-Aldrich, Inc (St Louis, Mo.) (“Sigma-LSRCCC”) and, for example, The Plant Culture Catalogue and supplement also from Sigma-Aldrich, Inc (St Louis, Mo.) (“Sigma-PCCS”), all of which are incorporated herein by reference.

The culture medium to be used should in a suitable manner satisfy the demands of the respective strains. Descriptions of culture media for various microorganisms are present in the “Manual of Methods for General Bacteriology” of the American Society for Bacteriology (Washington D.C., USA, 1981), incorporated by reference herein.

The synthesized cells may be cultured continuously, or discontinuously in a batch process (batch cultivation) or in a fed-batch or repeated fed-batch process for the purpose of producing the desired organic compound. A summary of a general nature about known cultivation methods is available in the textbook by Chmiel (Bioprozeßtechnik. 1: Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)), all of which are incorporated by reference herein.

Classical batch fermentation is a closed system, wherein the composition of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art.

Continuous fermentation is a system where a defined fermentation medium is added continuously to a bioreactor and an equal amount of conditioned medium is removed simultaneously for processing and harvesting of desired biomolecule products of interest. Continuous fermentation may maintain the cultures at a constant high density where cells are primarily in log phase growth. Continuous fermentation systems strive to maintain steady state growth conditions.

Methods for modulating nutrients and growth factors for continuous fermentation processes as well as techniques for maximizing the rate of product formation are well known in the art of industrial microbiology.

For example, a non-limiting list of carbon sources for cellular cultures include sugars and carbohydrates such as, for example, glucose, sucrose, lactose, fructose, maltose, molasses, sucrose-containing solutions from sugar beet or sugar cane processing, starch, starch hydrolysate, and cellulose; oils and fats such as, for example, soybean oil, sunflower oil, groundnut oil and coconut fat; fatty acids such as, for example, palmitic acid, stearic acid, and linoleic acid; alcohols such as, for example, glycerol, methanol, and ethanol; and organic acids such as, for example, acetic acid or lactic acid.

A non-limiting list of the nitrogen sources include, organic nitrogen-containing compounds such as peptones, yeast extract, meat extract, malt extract, corn steep liquor, soybean flour, and urea; or inorganic compounds such as ammonium sulfate, ammonium chloride, ammonium phosphate, ammonium carbonate, and ammonium nitrate. The nitrogen sources can be used individually or as a mixture.

A non-limiting list of the possible phosphorus sources include, phosphoric acid, potassium dihydrogen phosphate or dipotassium hydrogen phosphate or the corresponding sodium-containing salts.

The culture medium may additionally comprise salts, for example in the form of chlorides or sulfates of metals such as, for example, sodium, potassium, magnesium, calcium and iron, such as, for example, magnesium sulfate or iron sulfate.

Finally, essential growth factors such as amino acids, for example homoserine and vitamins, for example thiamine, biotin or pantothenic acid, may be employed in addition to the abovementioned substances.

In some embodiments, the pH of the culture can be controlled by any acid or base, or buffer salt, including, but not limited to sodium hydroxide, potassium hydroxide, ammonia, or aqueous ammonia; or acidic compounds such as phosphoric acid or sulfuric acid in a suitable manner. In some embodiments, the pH is generally adjusted to a value of from 6.0 to 8.5, preferably 6.5 to 8.

The cultures may include an anti-foaming agent such as, for example, fatty acid polyglycol esters. The cultures may be modified to stabilize the plasmids of the cultures by adding suitable selective substances such as, for example, antibiotics.

The cultures may be carried out under aerobic or anaerobic conditions. In order to maintain aerobic conditions, oxygen or oxygen-containing gas mixtures such as, for example, air, are introduced into the culture. It is likewise possible to use liquids enriched with hydrogen peroxide. The fermentation is carried out, where appropriate, at elevated pressure, for example at an elevated pressure of from 0.03 to 0.2 MPa. The temperature of the culture is normally from 20° C. to 45° C. and preferably from 25° C. to 40° C., particularly preferably from 30° C. to 37° C. In batch or fed-batch processes, the cultivation may be continued until an amount of the desired product of interest (e.g. an organic-chemical compound) sufficient for recovery has formed. This aim can normally be achieved within 10 hours to 160 hours. In continuous processes, longer cultivation times are possible. The activity of the microorganisms results in a concentration (accumulation) of the product of interest in the fermentation medium and/or in the cells of said microorganisms.

Algorithmic Enzyme Selection

Overview

Embodiments of the disclosure provide an algorithmic, computer-implemented approach to select enzymes as candidates for catalyzing a reaction. This approach substantially reduces the time required to determine optimal enzymes and eliminates human error. It also enables continuous improvement of prediction accuracy via refinement of predictive models based on the empirical data generated as a result of experimental validation of the sets of selected sequences.

Because of the ability to handle enormous data sets, embodiments employing algorithmic biological sequence selection may cause an exponential increase in potential candidate sequences. Embodiments of the disclosure address this issue by performing clustering or alternative path elimination (or both) to refine the selection of candidate sequences while maintaining the diversity of the sequence space.

Moreover, embodiments of the disclosure enable the identification of sequences that are statistically more similar to the desired function than manual approaches that rely on the functional human annotation of sequences.

More generally, embodiments of the disclosure may select biological sequences for enabling the performance of a desired biological function, e.g., in a host cell. In addition to enzymes, such sequences may include, for example, transporters, transcription factors, and nucleic acid sequences that code for proteins such as enzymes for catalyzing reactions. In addition to an enzymatic reaction, functions may include facilitation or regulation of cellular processes such as gene transcription/translation, transport of molecules across membranes, and stabilization or degradation of molecules.

Embodiments of the disclosure identify candidate biological sequences for enabling a function based upon sequences that are known or believed to enable the same or a similar function in different cells. The cells may, for example, be found in different species. In other cases, different sequences that carry out the same function in the same species, however, may exhibit different attributes that a scientist would find desirable for one purpose but not another.

Operation

In embodiments of the disclosure, the engine includes program code for identifying a candidate biological sequence for enabling a function in a host cell. The engine may: access a predictive model that associates a plurality of biological sequences with one or more functions; predict, using the predictive model, that one or more candidate sequences of the plurality of biological sequences enable a desired function in the host cell; and classify candidate sequences that satisfy a confidence threshold as filtered candidate sequences. In embodiments, the biological sequences are enzymes for catalyzing reactions (the function being the enzyme-catalyzed reaction). The engine may provide to a gene manufacturing system information concerning a first filtered candidate sequence, so that the gene manufacturing system may use the first filtered candidate sequence to produce a desired molecule.

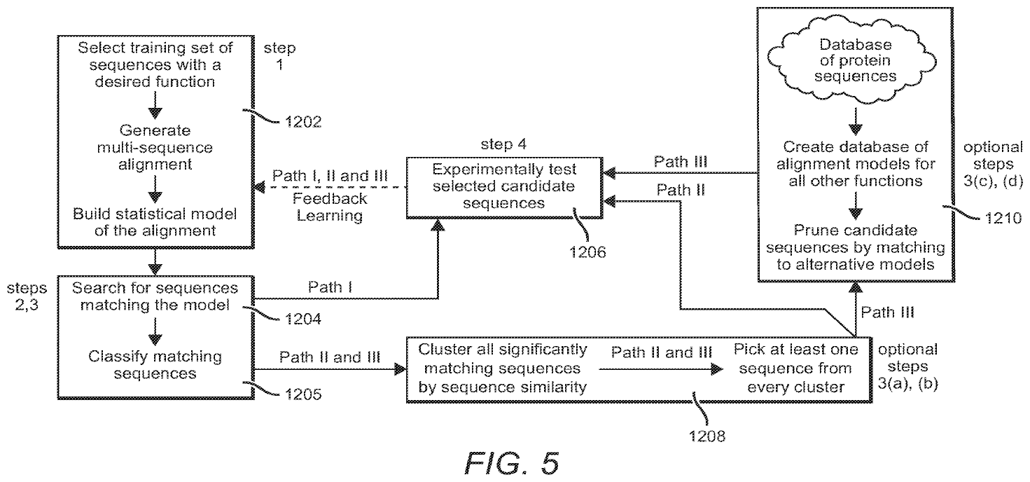

FIG. 5 is a flow diagram illustrating the operation of embodiments of the disclosure. Unless otherwise indicated, these operations may be performed by software residing in the engine. Although the description below concerns the identification of enzyme amino acid sequences, the same approach may be used to identify other sequences, as noted below.

According to embodiments of the disclosure, the engine may perform the following operations:

Step 1 1202: Obtaining the Predictive Model

The engine may generate (or retrieve from an internal or external database) one or more models trained on instances of enzymes physically verified, or predicted with a high degree of confidence, to carry out the desired function. Examples of functions are: enzymatic activity such as tyrosine decarboxylase, which is an enzyme that catalyzes the conversion of tyrosine to tyramine; and alpha-amylase, which is an enzyme that catalyzes the hydrolysis of alpha-bonds in complex polysaccharides.

Instead of enzymes, embodiments of the disclosure may identify nucleic acid sequences that code for enzymes of interest. Further, functions represented by such models are not limited to enzymes of metabolic reactions, however, and may also, for example, refer to functions, such as DNA helicases, which are responsible for separating two strands of DNA or proteins, and other non-catalytic types of functions such as transcription factors, transporters, structural proteins, as well as nucleotide sequences that are not translated into peptides such as transfer RNAs, and small non-coding RNAs. In addition, one or multiple models can be generated for each functional activity that abstracts diversified information such as phylogeny, orthology, sequence similarity, enzyme subunits, and protein morphology.

The term “models” here includes but is not limited to statistical models such as

Hidden Markov Models (HMIs), dynamic Bayesian networks, artificial neural networks (ANNs) including recurrent neural networks such as those based on Long Short Term Memory Models (LSTM) as well as derivatives and generalizations thereof, and other machine learning-based models.

As an example of a predictive model, for step 1, the engine may rely on HMM, which is a statistical model of multiple sequence alignments (MSAs). In bioinformatics, a sequence alignment is a way of arranging the sequences such as DNA, RNA, or protein, to identify regions of similarity that may be a consequence of functional, structural, and/or evolutionary relationships among the sequences. In evolutionary biology, conserved sequences are similar or identical sequences in nucleic acids (DNA and RNA) or proteins across species (orthologous sequences) or within a genome (paralogous sequences). Conservation indicates that a sequence has been maintained by natural selection. Amino acid sequences can be conserved to maintain the structure or function of a protein or domain.

As an example of finding a protein amino acid sequence for a reaction (function), which may be part of the reaction pathway output of embodiments above, the engine may retrieve from database 110 a training set of enzymes that catalyze the reaction. Each enzyme may be found in a different species. However, not every amino acid in the enzymes is important to performing the function. The observed frequency with which an amino acid occupies the same position in different enzyme sequences that perform the same function (the degree to which the amino acid is “conserved”) correlates to the likelihood that the amino acid enables performance of that function. This is the basis for using an MSA to identify other enzyme sequences for performing a desired function. The engine employing an MSA model provides the output sequences along with a measure of the degree of confidence (based on the conservation of the sequences) that a sequence enables the desired function.

Conserved sequences may be identified by homology search, using tools such as BLAST, HMMER and Infernal. Homology search tools may take an individual nucleic acid or protein sequence as input, or use statistical models generated from multiple sequence alignments of known related sequences. Statistical models, such as profile-HMMs, and RNA covariance models which also incorporate structural information, can be helpful when searching for more distantly related sequences. Input sequences are then aligned against a database of sequences from related individuals or other species. The resulting alignments may then be scored based on the number of matching amino acids or bases, and the number of gaps or deletions generated by the alignment. Acceptable conservative substitutions may be identified using substitution matrices such as PAM and BLOSUM. Highly scoring alignments are assumed to be from homologous sequences. The conservation of a sequence may then be inferred by detection of highly similar homologs over a broad phylogenetic range.

Identifying conserved sequences can be used to discover and predict functions of sequences such as proteins and genes. Conserved sequences with a known function, such as protein domains or motifs, can also be used to predict the function of a sequence. Databases of conserved protein domains or motifs such as Pfam and the Conserved Domain Database can be used to annotate functional domains or motifs of predicted proteins.

Example Inputs and Outputs

Input step 1: an enzymatic activity/reaction from a predicted pathway/pedigree, such as “tyrosine decarboxylase,” which may be represented by the chemical equation “L-Tyrosine<=>Tyramine+CO2,” and a training set of sequences that are believed to have this enzymatic activity/catalyze this reaction (e.g., based on scientific publications, experimental data from a public or internal database or a computational prediction based on homology to sequences with experimental evidence of the required activity).

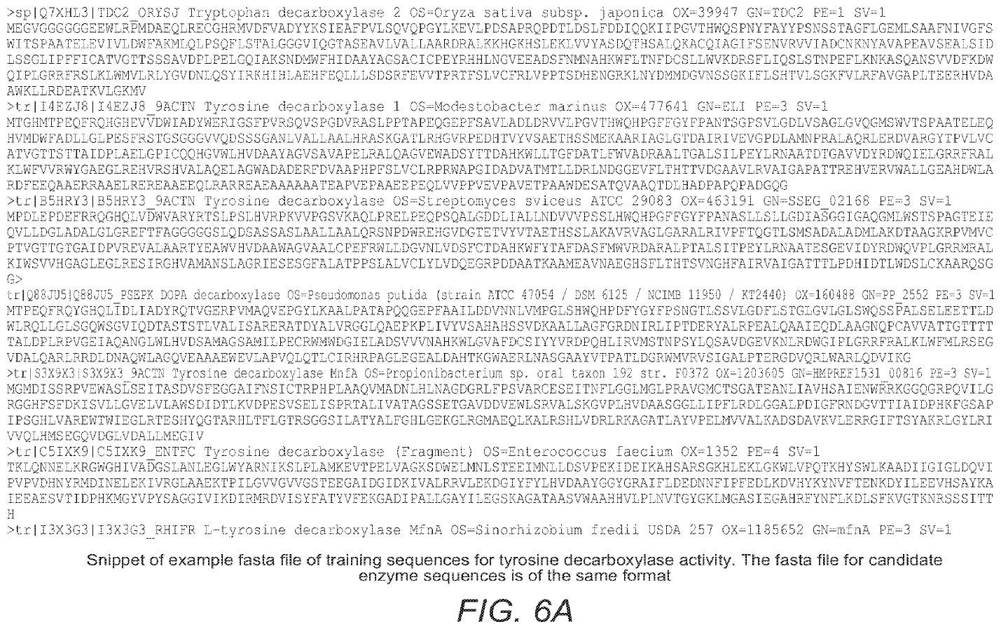

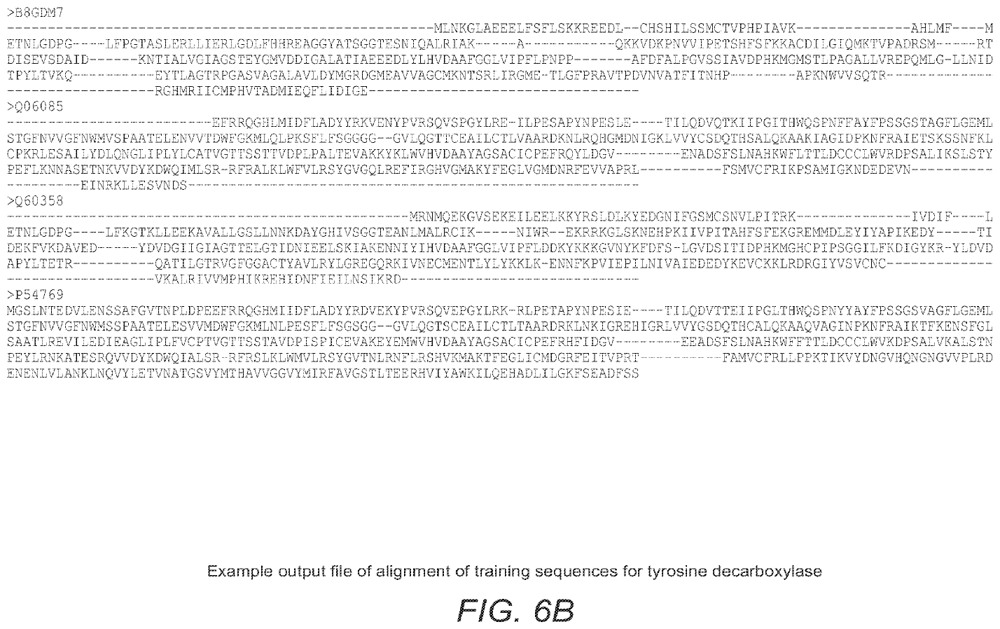

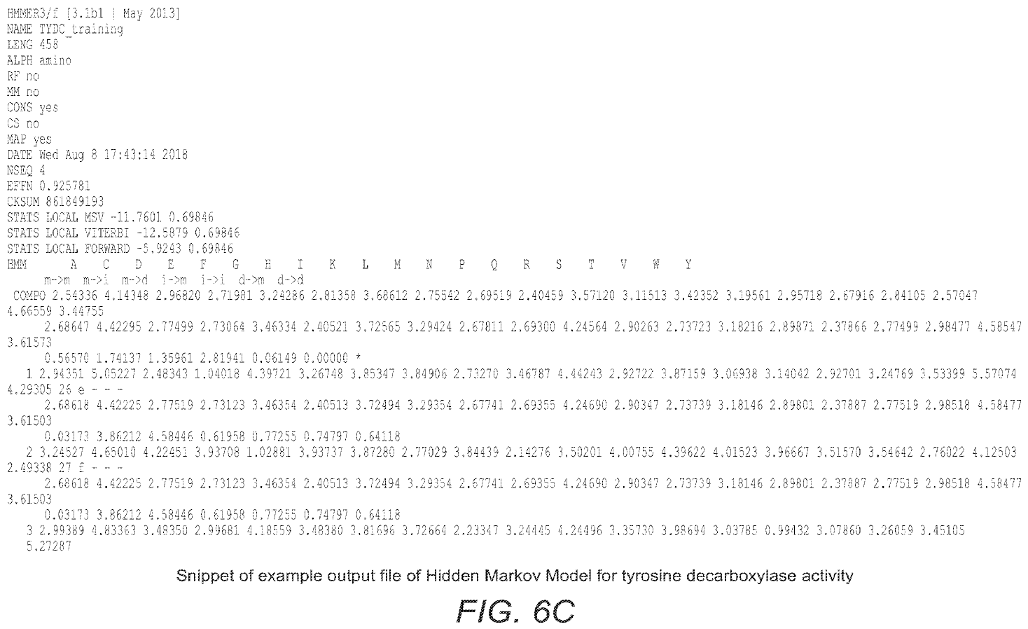

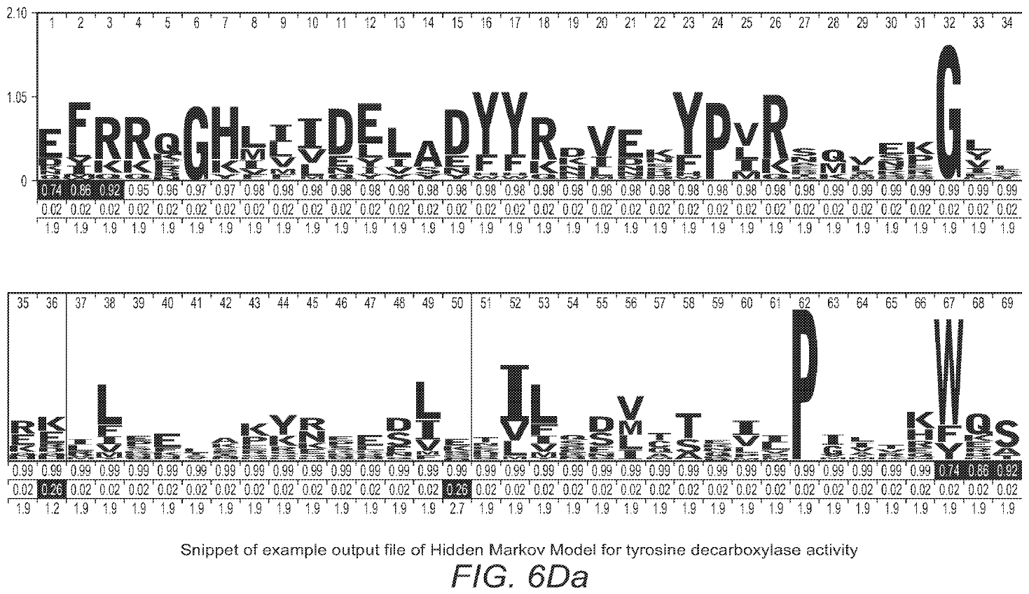

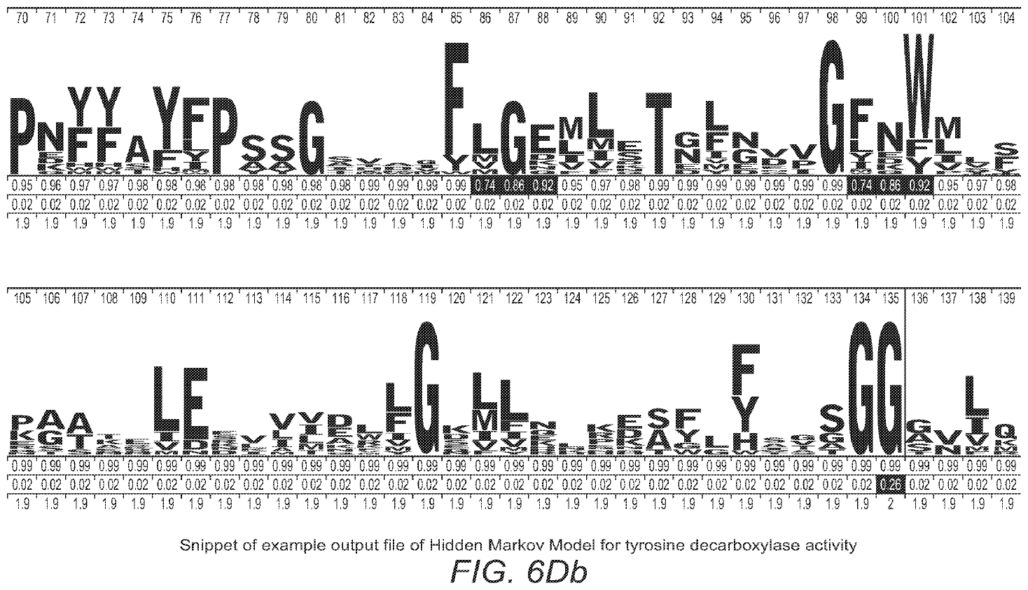

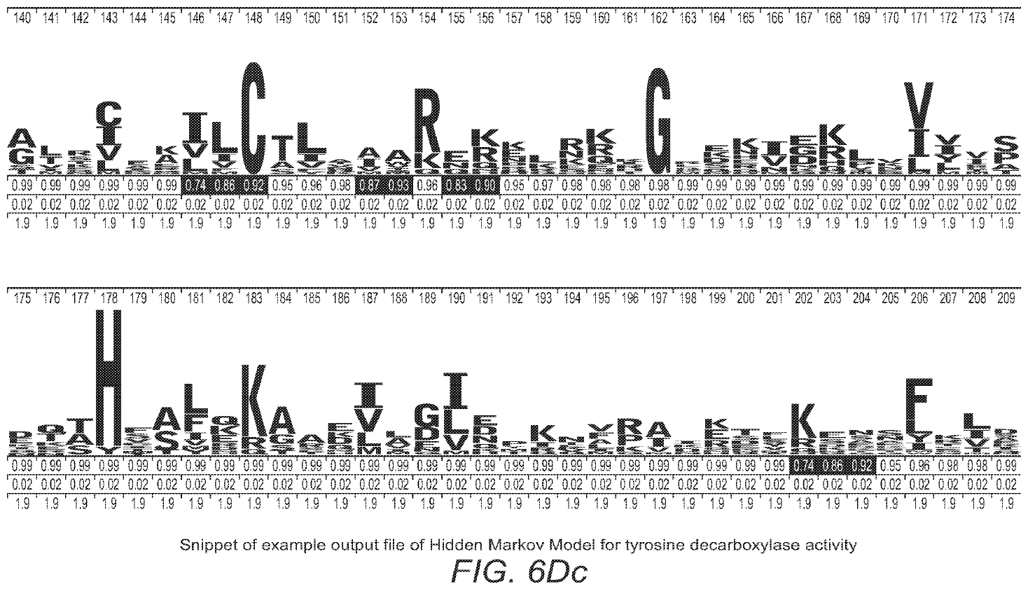

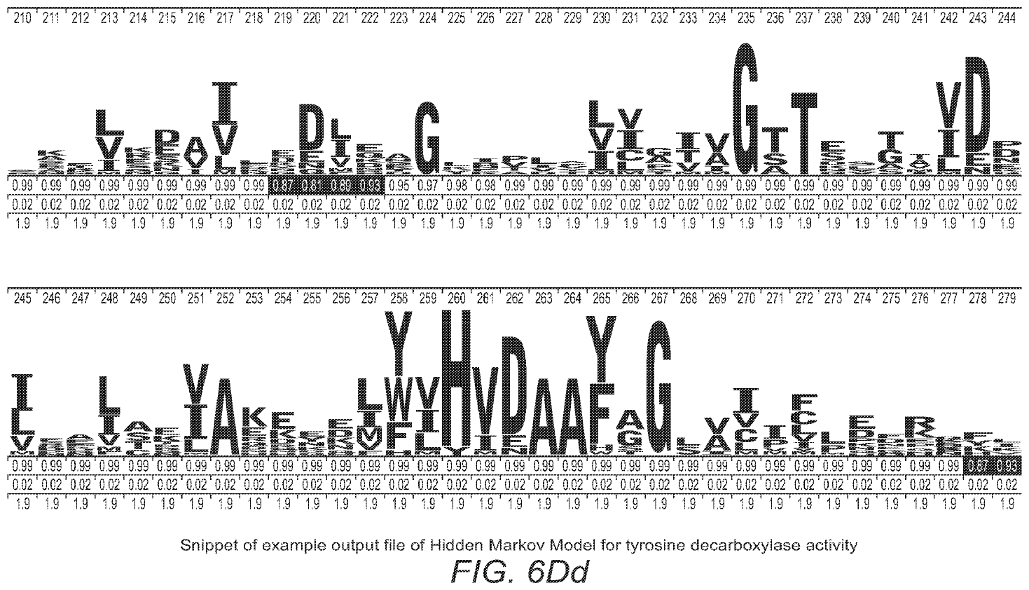

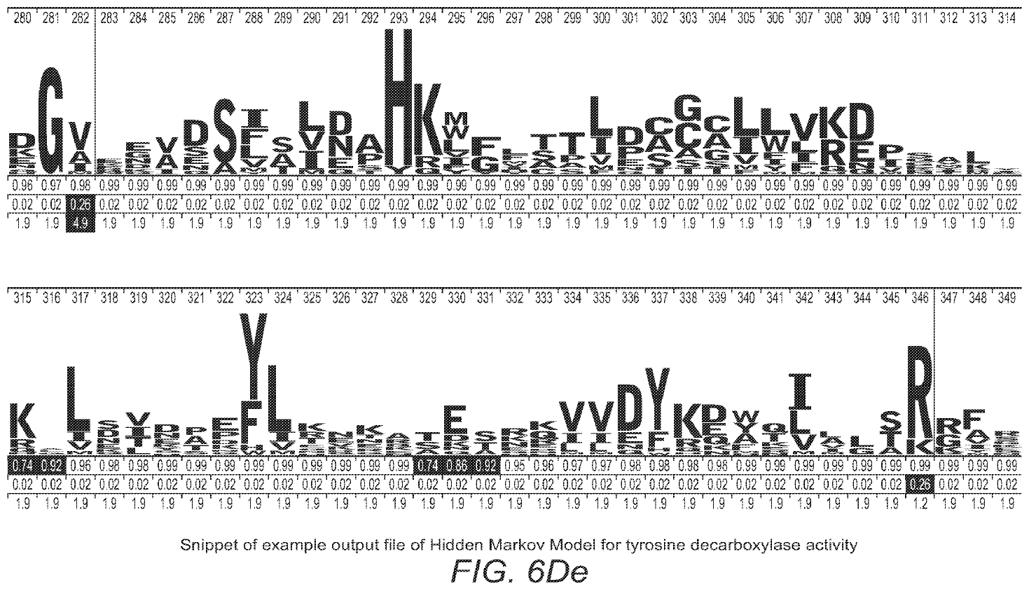

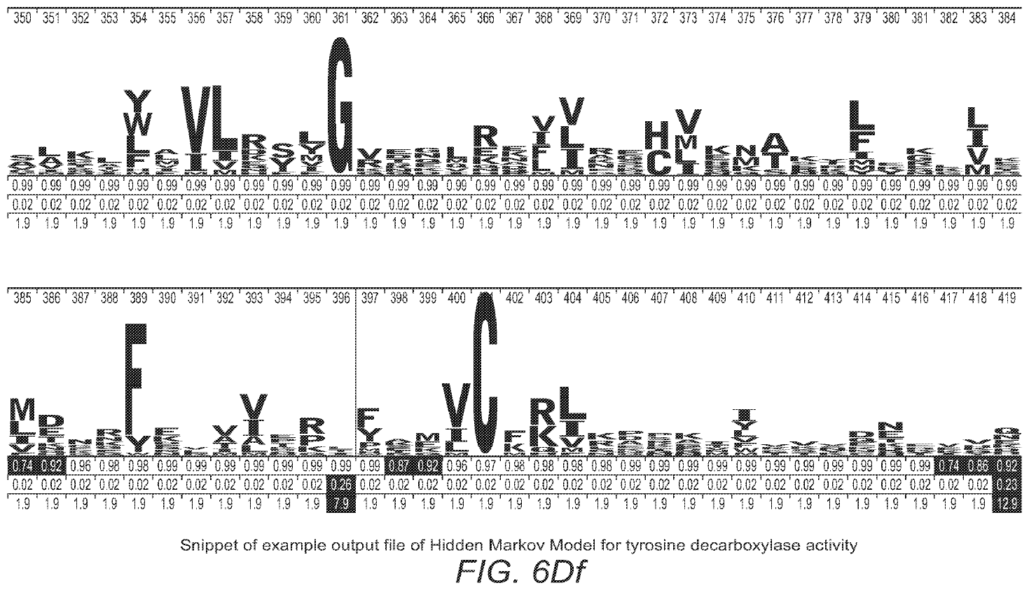

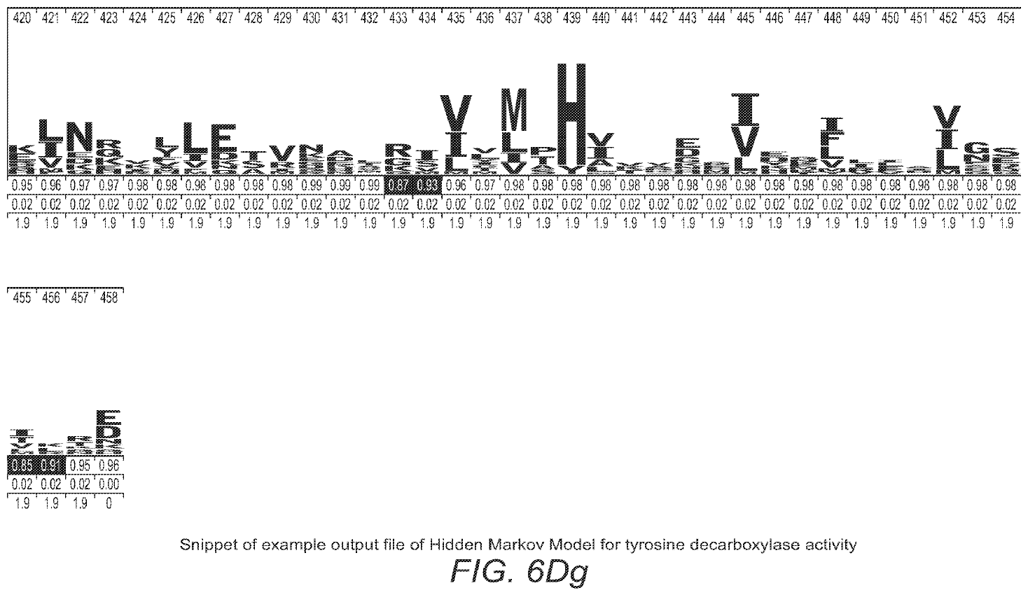

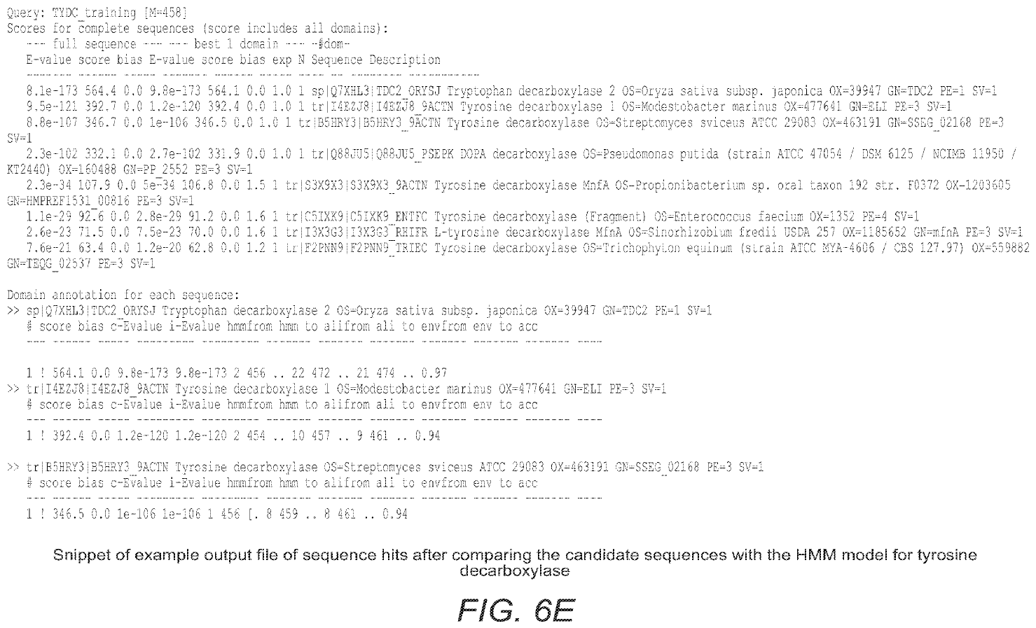

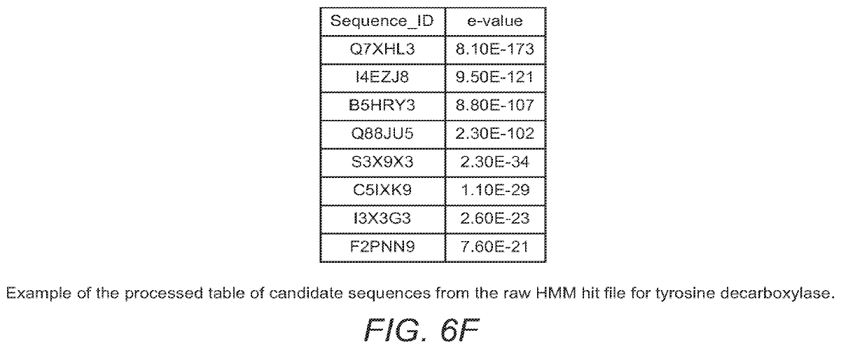

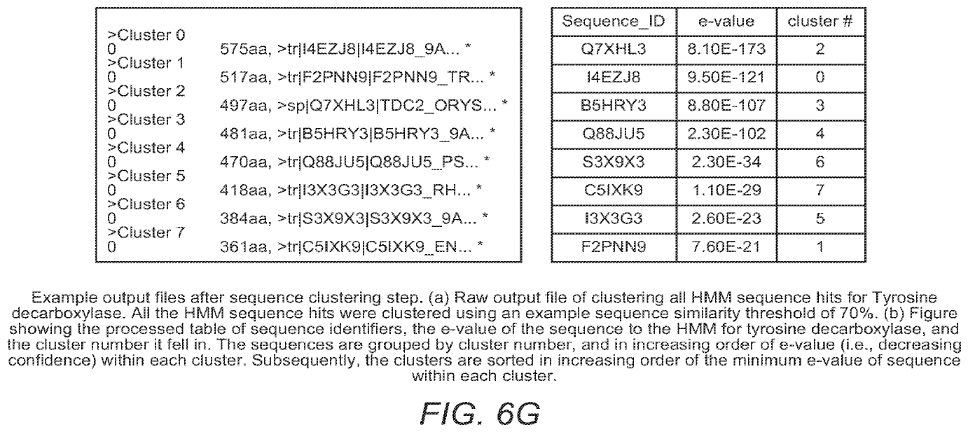

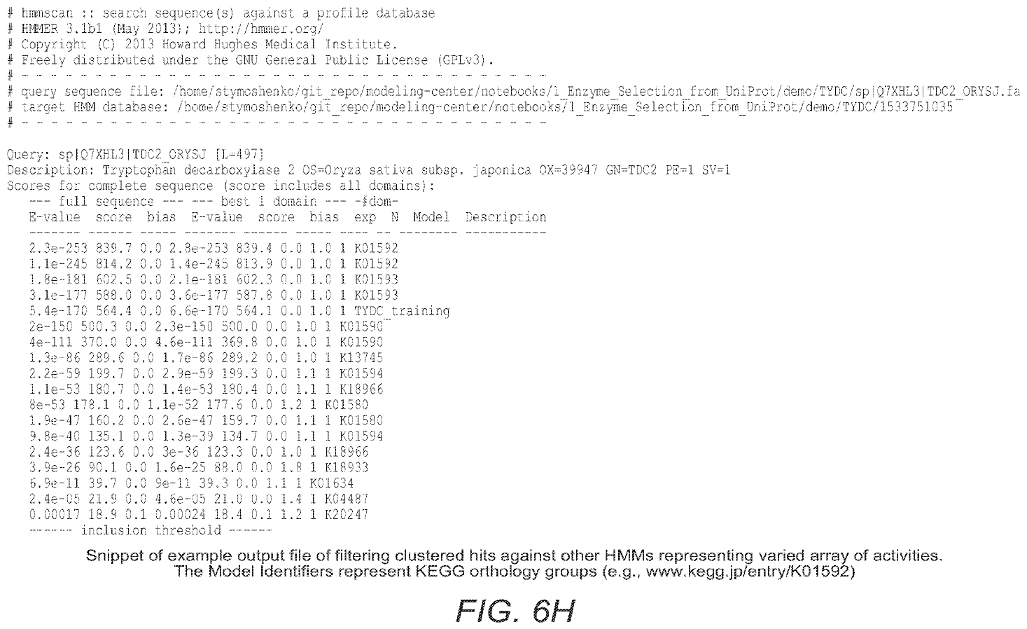

FIGS. 6A-6H illustrate a prophetic example of identifying at least one sequence to enable tyrosine decarboxylase activity using the HMMER tool, according to embodiments of the disclosure. One of ordinary skill in the art would understand how to interpret these figures, especially in view of Eddy, et al., HMMER User's Guide: Biological sequence analysis using profile hidden Markov models, Version 3.1b2; February 2015, incorporated by reference herein in its entirety.

FIG. 6A illustrates a snippet of an example FASTA file containing a training set of enzymes that catalyze for tyrosine decarboxylase activity. The file contains the amino acid sequences of the training set of enzymes encoding for the reaction activity. Note that the annotations in the file indicate activity other than tyrosine decarboxylase, such as tryptophan decarboxylase, because the displayed annotations were derived from a commercially available database. However, embodiments of the disclosure determined that such sequences, in fact, enabled tyrosine decarboxylase activity. Thus, embodiments of disclosure enable correct recordation of annotations in otherwise incorrect publicly available databases.

Output step 1: multi-sequence alignment(s) of the sequences present in the training set and a model (or multiple models) representative of this alignment, including an indicator of the degree of confidence that a unit within the sequence (e.g., an amino acid) is related to the desired function (e.g., expectation value, probability that the unit is conserved at a given position within the sequence). FIG. 6B shows snippet of an output file showing such a multi-sequence alignment of the training set of enzymes encoding for a tyrosine decarboxylase reaction. An identifier (e.g., B8GDM7) following the “>” sign identifies an enzyme sequence, and the text below shows the corresponding sequence. In this example, spaces, as indicated by “−” in the amino acid sequences, indicate positions where a particular enzyme sequence does not align with the consensus alignment of all enzymes in the training set of enzymes. The consensus alignment is determined by optimal subsequences that are conserved, through similarity and/or identity, across all the sequences in the training set of enzymes.

FIG. 6C shows a snippet of an output file of a Hidden Markov Model (using the HMMER tool) constructed from the multi-sequence alignment file shown in FIG. 6B, from which a skilled artisan can determine the degree of confidence that an amino acid within the sequence is related to the desired tyrosine decarboxylase activity (function). FIG. 6D shows a pictorial representation of the same statistical model for tyrosine decarboxylase activity, where the height of the each amino acid annotation represents the propensity of that particular amino acid in that position (represented on the x axis) to be related to the desired function of the overall enzyme.

Step 2 1204: Matching Database of Sequences to Model

The engine may perform a search for candidate sequences for enabling the function of interest using the model(s) trained in step 1, by comparing every sequence in a source database (such as Uniprot, KEGG, NCBI, JGI GOLD or a proprietary database of nucleotide or protein sequences) to the model(s) generated in step 1. Examples of the tools that could be used for this process is HMMsearch, HMMscan, or Recurrent Neural Networks designed for search by LSTM models.

Example Inputs and Outputs

Input step 2: the model(s) trained on the trusted set(s) of sequences with the desired function and a search database of sequences.

Output step 2: due to the size of the source databases, the engine may output a set of sequences ranging from a few to hundreds of thousands (for just one reaction) that significantly match (with a high probablility score) to the model(s) produced in step 1. FIG. 6E shows a snippet of example output file of sequence hits after comparing the candidate sequences with the HMM model for tyrosine decarboxylase. In this example file, the confidence of a particular enzyme sequence from a database matching to the HMM of tyrosine decarboxylase is enumerated by the E-value metric. The lower the E-value of an enzyme, the higher the statistical confidence of a match to the model.

FIG. 6F shows an example of the processed table of candidate sequences from the raw output file for FIG. 6E that extracts the identifier of the sequence from the search database and the E-value of the match to the tyrosine decarboxylase HMM model sorted in ascending order of E-value. In this example, the enzyme sequence Q7XHL3 has the lowest E-value, and thus is ranked as the amino acid sequence most likely to enable tyrosine decarboxylase activity.

Embodiments of the disclosure provide further refinements to reduce the size of this potentially enormous data set.

Step 3 1205: Filtering Matching Sequences

The engine may classify the candidate sequences from step 2 based on threshold parameters (e.g., minimal probability score such as expect value (E-value) or significance threshold) that may be determined by the user or another based on the intended purpose and trade-offs between precision and scope of the search. For example, assume step 2 results in a large number of sequences that enable the desired function with low degrees of confidence. In such cases, a user may adjust a first confidence threshold so that the engine eliminates sequences that do not satisfy that first threshold to result in a more manageable number of candidate sequences with higher confidence. The candidate sequences that satisfy the first confidence threshold (surviving step 3) may be referred to as “filtered candidate sequences” if the workflow follows Path I, shown in FIG. 5 and described below. If Path II or Path III is taken, then the candidate sequences that enter step 4 from optional step 3(b) or 3(d), respectively, may be referred to as “filtered candidate sequences.”

For example, depending on the size of the training set, size of the sequence database, and number of candidate sequences found at the step 2, as well as other factors, a user may set the minimal degree of confidence, e.g. expect-value, as permissive as 1E-10* or higher (to broaden the scope of the search by sacrificing precision), or, conversely, as strict as 1E-50** or lower to increase the precision with the caveat of a reduced scope.
*estimated one out of ten billion sequences in the target database would be a better match to the given model than the candidate sequence with the e-value 1E-10
**estimated one out of 10⁵⁰ sequences in the target database would be a better match to the given model than the candidate sequence with the e-value 1E-50.

Example Inputs and Outputs

Input step 3: One or More Sequences that Match the Model(s) Representing the Function of Interest

Output Step 3: A subset of (filtered) candidate sequences that match the model(s) representing the function of interest and satisfy a user-defined minimal, first degree of confidence threshold.

Step 4 1206: Refining Predictive Model

The candidate sequences that satisfy the first confidence threshold in step 3 may be synthesized and tested to ascertain empirically if they catalyze the desired function as predicted by the model. (The same operations may be performed on the candidate sequences resulting from optional Paths II and III, which are described below.) This test can be performed as an in vitro enzyme assay, or via incorporation of the sequences into host(s) through, but not limited to, chromosomal integration or replicated plasmids. For those sequences that produced the desired function under the particular experimental conditions, the engine may record the result in the model database (e.g., database 110). For those sequences where the desired function was not detectable, engine may also record that result in the database 110. The engine may use these records to expand/refine the set of training sequences for the model(s) representing this function as the “positive” and “negative” training set/examples.

According to embodiments of the disclosure, the engine repeats steps 1-4 (and steps 3(a)-(d), to the extent those options are chosen) for each reaction (e.g., in a pathway leading to a desired molecule), and stores the results in database 110.

A change in the experimental setting (such as a change in the host cell or growth media) may change the empirical outcomes. For example, not all sequences may produce the desired function in all possible conditions. The engine may record this result in the database 110 such that subsequent searches with the same combination of host and experimental conditions would exclude the negative examples.

The number of sequences chosen to be validated experimentally may be limited by available throughput. In a high-throughput factory-like setting, in principle, many sequences could be tested simultaneously for the same functionality. The “re-training,” via feedback loop, of the models based on positive and negative outcomes observed enhances the predictive power and precision of the models with every select-test-retrain cycle (illustrated as part of Paths I, II and III in FIG. 5). To this end, automated, high-throughput experiments can yield large and consistent training sets, thereby enabling retraining in a consistent manner that is robust to occasional errors and biological variability.

Example Inputs and Outputs

Input Step 4: Candidate Sequences to be Validated

Output Step 4: Recorded Results of Experimental Validation in Database to Update Predictive Model

Optional Steps 3(a) and 3(b) 1208: Clustering

Referring to FIG. 5, steps 1, 2, 3 and 4 described above follow the arrows labeled with “Path I.” FIG. 5 also illustrates optional Paths II and III, which may be performed to further refine the filtered candidate sequences, according to embodiments of the disclosure. The candidate sequences resulting from Paths II and III, like those from Path I, are subject to step 4, according to embodiments of the disclosure.

Path II includes steps 3(a) and 3(b) 1208. In embodiments, the engine may (e.g., if the user elects) take additional steps 3(a) and 3(b) before step 4 to diversify the candidate sequences that satisfy the first confidence threshold.

Step 3(a) 1208: The predictive engine 109 may perform statistical clustering (based on, for example, sequence similarity, or t-Distributed Stochastic Neighbor Embedding) on the candidate sequences that satisfy the first confidence threshold. The engine may record which sequences are sufficiently similar to appear in the same cluster. For example, using the CD-HIT clustering algorithm, the engine may denote sequences as belonging to the same cluster if they exceed a 38%-99% sequence identity threshold. This value is a user-defined parameter that reflects the maximal degree of identity among the sequences, which a user allows to include in the final filtered set of candidates. In the left table, FIG. 6G shows a snippet of the raw output file resulting from clustering all HMM sequence hits for tyrosine decarboxylase. All the HMM sequence hits are clustered using an example sequence identity threshold of 70%. The figure shows a snippet of the file that lists the cluster number and the sequence identifiers of all the sequences that lie within that cluster. (In this snippet, the full list of sequence identifiers is truncated as indicated by the asterisks.) In this manner, a user can address the challenge of evenly exploring candidate sequences when their number exceeds the experimental capacity for testing all the candidates.

Optional Step 3(b) 1208: Selecting Sequence(s) from the Clusters

The engine may select one or more sequences from each cluster. The number of sequences selected may depend upon the number of clusters, which in turn depends on the user-defined sequence identity threshold as well as the overall “sequence diversity” within the set of candidate sequences prior to the clustering. Selection of a particular candidate sequence(s) from each cluster may be informed by the degree of confidence (e.g. the e-value of the match to the corresponding model). This ensures that not only a diversified set of candidates are selected for each function/reaction but also that the candidates with the highest likelihood of desired function are prioritized. FIG. 6G (right table) shows the example processed table output of sub-selected sequences where only the sequence with lowest e-value is selected from each cluster, after clustering step 3(a). The table shows the identifiers of those enzymes, the e-value of the sequence matching to the HMM for tyrosine decarboxylase, and the cluster number in which it fell, which is generated by parsing the output file in the left table of the figure. The right table shows the sorted sequences by increasing e-value (i.e., decreasing confidence).

Optional Steps 3(c) and 3(d) 1208: Eliminating Candidate Sequences That Have Affinity Toward Alternative Functions

Path III includes steps 3(c) and 3(d) 1210. In embodiments, the engine may (e.g., if the user elects), take additional steps 3(c) and 3(d) before step 4 to reduce the likelihood that the candidate sequences that satisfy the first confidence threshold represent undesired functions. In embodiments, steps 3(c) and 3(d) may be chosen only if the confidence scores of the candidate sequences that satisfy the first confidence threshold are above or below a second threshold.

Optional Step 3(c): Creating Data Set of Models for Other Functions

In embodiments, the engine may prepare a database of predictive models that represent all known functions for which such model(s) can be constructed, e.g., all KEGG orthology groups that are associated with at least one sequence that has been empirically observed to carry out a corresponding function.

Optional Step 3(d): Eliminating Candidate Sequences That Have Affinity Toward Alternative Functions

In embodiments, the engine may prevent classification, as a filtered candidate sequence, of a candidate sequence that satisfies the first confidence threshold but that is more likely, within a given tolerance (e.g. between 0.5 and 1, where 1 represents no tolerance to the possibility of an alternative function), to enable a function different from the desired function. To do so, the engine may compare (e.g,. using HMMscan) each candidate sequence resulting from step 3 (satisfying the first confidence threshold, e.g., 0.8) to each of the models stored in the database in step 3(c), to find and eliminate sequences that have a higher confidence score (given the tolerance parameter) for any function other than the desired function. FIG. 6H shows a snippet of an example output file of filtering clustered hits against other Hidden Markov Models representing a varied array of reaction activities. In this example, the Model Identifiers represent KEGG orthology groups that represent a particular reaction activity. For each identified sequence, the figure shows the expectation-value with which the sequence matches to the HMMs in the scanning database of different activities. The expectation score of the identified sequence to the desired activity (tyrosine decarboxylase shown as TYDC_training) in relation to those of other activities quantifies how specific is the sequence to the desired activity. For example, for the sequence Q7XHL3, the desired tyrosine decarboxylase activity is not the activity with the least e-value, and hence, may not be the best candidate sequence to test.

A user-defined tolerance parameter may be used to set a limit as to how much the confidence that a candidate sequence produces a desired function is allowed to fall below a confidence that it also produces an undesired function. The engine may compare the confidence that a given candidate sequence enables a desired function to the confidence levels that the candidate sequence enables any other known functions stored in a database, according to their predictive models. This tolerance parameter allows the user to address cases where a candidate sequence may be predicted to match multiple functions (represented by models) with varying degrees of confidence, and the user would like to ensure that the model representing the desired function is one of the best matches (if not the best match) for the candidate sequence. For example, this tolerance can be a ratio of the (log of the lowest e-value found when compared to the database of all models) divided by the (log of the e-value when compared to the model representing the desired function). In that instance, if the best-matching model is also the one representing the desired function, the ratio will be 1. In all other cases, the ratios lower than 1 would denote decreased confidence about the given candidate sequence having the desired function and not the function represented by the model which is the best match (e.g., the once with the lowest e-value).

Example Based on Experimental Data

Using the sequence selection process essentially as illustrated by FIG. 5, path III (i.e., all the steps except the feedback learning), between 48 and 72 candidate sequences were selected for 3 enzymatic functions of interest from a meta-genomic collection of protein sequences. In the same manner 72 candidate sequences were also selected for a small-molecule exporter function of interest. Notably, all four functions were native to the microbe in which selected sequences were tested, but were deemed of interest based on the assumption that they may be limiting for production of the target molecule or its export from the cells.

Each one of the selected protein sequences was back-translated into a coding DNA sequence, synthesized and inserted in the genome of the microbe, which was already a highly-effective industrial producer of the molecule of interest. These modified microbes were tested for the improvement in production of the specific molecule in terms of two phenotypes of interest: (1) speed of production in gram per L per hour; (2) overall substrate-to-product conversion efficiency in gram per gram. Multiple sequences representing two of the three enzymatic functions and one exporter function resulted in a statistically significant improvement of over 1% for at least one of the two phenotypes of interest. In such a highly-optimized, industrially-used microbe it would be rare to observe any change that improved one of the phenotypes without a detrimental effect on the other one. Nevertheless, multiple of the candidate sequences conferred such an improvement. To measure phenotypic improvement, each of the algorithmically-selected sequences was engineered individually into the host microbe, and then the resulting phenotypic improvement was evaluated.

This experiment demonstrated utility of the workflow illustrated by FIG. 5 for finding highly efficacious candidate sequences for enzymatic and exporter functions even from a large meta-genome that consists of only predicted protein sequences without any functional annotations. The improvements in this example were obtained without the feedback learning of embodiments of the disclosure. Thus, one would expect feedback learning to result in prediction of sequences with even greater improvement.

Further Refinements on Selecting Candidate Biological Sequences for Screening

Embodiments of the disclosure described below provide additional refinements to reduce the size of the potentially enormous set of candidate sequences.

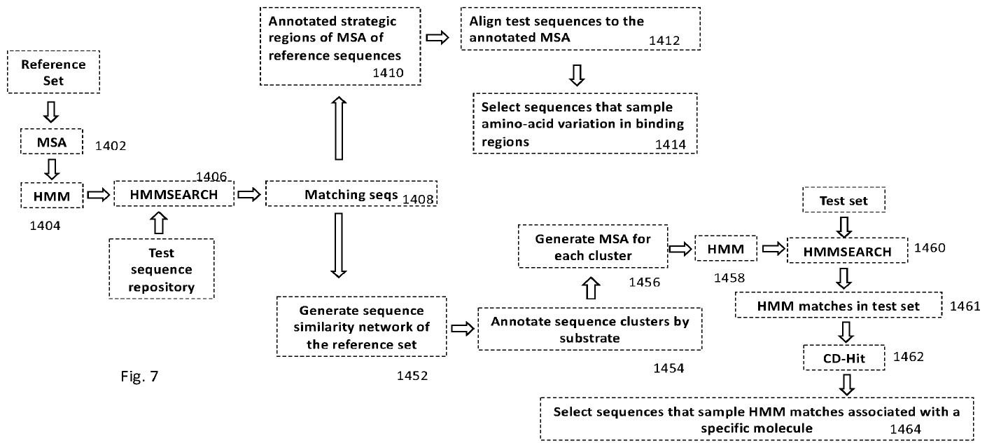

FIG. 7 illustrates two different approaches for selecting biological sequences for screening to determine whether they perform a desired biological function. According to embodiments of the disclosure, a statistical model is used to determine the degrees of similarity between test sequences and reference sequences that are known to perform the desired function. Examples of an appropriate statistical model include a Hidden Markov Model (HMM), a dynamic Bayesian network, an artificial neural network (ANN) including a recurrent neural network such as that based on Long Short Term Memory Models (LSTM) as well as derivatives and generalizations thereof, and other machine learning-based models.

When using an HMM, the engine may first create a multiple sequence alignment (“MSA”) of the reference sequences (1402), generate an HMM based on the MSA (1404), and use a homology search algorithm such as hmmsearch in the HMMER software package to determine the degree of similarity between the test sequences and the HMM model (1406) to determine matching test sequences (1408) that satisfy a degree of similarity, such as an E-value of 10, that may be equivalent to a percent identity greater than 25% between the test sequences and the reference sequences represented by the HMM model. In general, the three most popular measures for the statistical significance of protein sequence similarity are (1) percent identity, (2) E-value, and (3) bit-score. All these are correlated with each other: The higher the % ID, the higher the bit score, and the lower the E-value. Each statistical measure is the result of abstracting different features of similarity, hence we use them interchangeably in this document.

Clustering Sequences by Molecule/Substrate

Referring to FIG. 5, in step 1208, the engine may cluster candidate test sequences, and select at least one sequence from each cluster for diversification. According to embodiments of the disclosure with reference to FIG. 7, an alternative approach is used to determine similarities between reference sequences (e.g., from a training set) that are known to enable a biological function (whether in vivo or in vitro) and cluster the reference sequences based upon their similarities (1452), where each cluster corresponds to a molecule in that the cluster includes one or more of the reference sequences that are known to bind to the molecule, e.g., that bind with the molecule and catalyze a reaction involving the molecule (e.g., a substrate) as a reactant. The clustering may be performed using a well-known clustering technique, such as a sequence similarity network or CD-HIT.

According to embodiments of the disclosure, the engine compares test sequences with reference sequences in one or more clusters (1460) to determine test sequences that match the reference sequences (1461) within a satisfactory degree of similarity, e.g., 25% or more.

The engine may then select candidate test sequences that each correspond to a cluster (i.e., the test sequences match well with reference sequences in a corresponding cluster). However, the number of matching test sequences may still be an unwieldy amount to screen. As an example, assume that 1000 test sequences match the HMM for a particular cluster over a desired similarity threshold. The engine may use a sequence clustering algorithm, such as CD-HIT, to reduce the candidate count to an acceptable number (1462). CD-HIT takes as inputs a set of protein sequences and a desired sequence similarity metric such as a percent sequence identity. For example, if a user wants to select 10 out of the 1000 matches for screening, the user may select a sequence similarity (percent ID in CD-HIT) that would cluster the 1000 matching test sequences into ten clusters, and then select one test sequence from each of the ten clusters as candidate sequences (1464). As the threshold (strictness) for the required sequence similarity increases, sequence clustering algorithm will generate fewer clusters.

The similarities between the reference sequences may be statistical estimates, e.g., of all-by-all pairwise sequence similarity, determined by known tools for determining statistical similarities between biological sequences, such as BLAST. The clustering may be performed using a known graph-based clustering operation, such as that in the well-known Cytoscape bioinformatics software platform for visualizing molecular interaction networks. The engine may employ a statistical model to determine the similarities between the test sequences and the reference sequences in the one or more clusters. Examples of an appropriate model include a Hidden Markov Model (HMM), a dynamic Bayesian network, an artificial neural networks (ANN) including a recurrent neural network such as that based on Long Short Term Memory Models (LSTM) as well as derivatives and generalizations thereof, and other machine learning-based models. When using an HMM, the engine may create a multiple sequence alignment (“MSA”) of reference sequences in selected clusters (1456), generate an HMI based on the MSA for each selected cluster (1458), and use a homology search algorithm such as hmmsearch® in the HMMER® software package to determine the degree of similarity (homology) between the test sequences and the HMM model (1460) to determine matching test sequences (1461).

Selecting Candidate Sequences Based on Strategic Regions

FIG. 7 also illustrates a second approach for identifying candidate sequences for screening. According to embodiments of the disclosure, the engine determines degrees of similarity (homology) between test sequences and reference sequences that are known to enable a biological function. For example, the engine may determine the overall similarity between the test and reference sequences. According to embodiments of the disclosure, reference sequences may be known to enable a biological function by virtue of being indicated in a database or an electronic storage medium as enabling the function.

As described above, according to embodiments of the disclosure, a statistical model, such as HMM or another model mentioned above, is used to determine the degrees of similarity. When using an HMM, the engine may first create a multiple sequence alignment (“MSA”) of the reference sequences (1402), generate an HMM based on the MSA (1404), and use a homology search algorithm such as hmmsearch in the HMMER software package to determine the degree of similarity (homology) between the test sequences and the HMM model (1406) to determine matching test sequences (1408).

According to embodiments of the disclosure, the engine compares regions in the test sequences to one or more reference regions, in one or more of the reference sequences, that are known to bind with a molecule (e.g., a substrate). According to embodiments of the disclosure, a reference sequence may be known to bind with a molecule by virtue of the reference sequence being indicated in a database or an electronic storage medium as a having a binding region comprising, e.g., one or more amino acids, which itself may be determined via empirical methods.

According to embodiments of the disclosure, the engine may first determine a set of matching test sequences including test sequences having degrees of similarity to the reference sequences that satisfy a similarity threshold (e.g., greater than 25% percent identity), and then compare regions of the matching test sequences with the one or more reference regions.

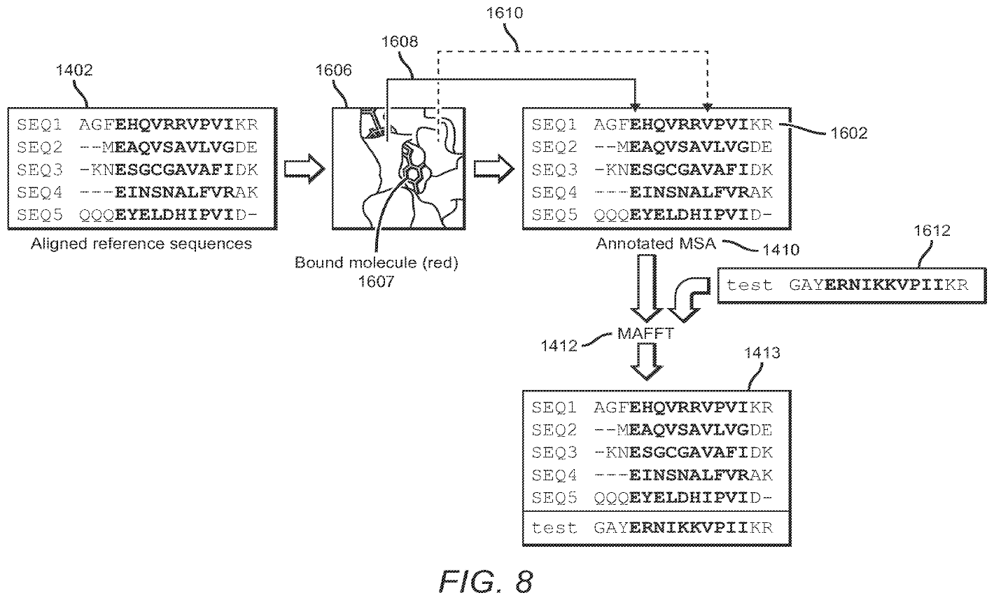

According to embodiments of the disclosure with reference to FIG. 8, a strategic region, such as a substrate binding site, in a reference sequence may be determined from an examination of the three-dimensional structure of the sequence using techniques such as selecting all amino acids that are a predetermined distance (e.g., 10 angstroms) away from the bound molecule. According to embodiments of the disclosure, a strategic region may also be determined computationally by selecting amino acids based on distances determined using software such as LIGPLOT, which computes the amino acid neighbors of a molecule given a crystal structure.

As an example, FIG. 8 shows a reference sequence Seq1 1602 that includes strategic regions 1608 and 1610, as determined from an examination of the 3D crystallographic structure 1606 of Seq1 1602. In the fourth position 1608, the first strategic region includes an amino acid E that is known to bind with a particular substrate 1607. In the tenth position 1610, the second strategic region includes an amino acid V in Seq1 1602, which also binds to substrate 1607. In this example, each region includes just one amino acid, although a region may include more than one amino acid. In general, a “strategic region” in a reference sequence (alternatively referred to herein as a “reference region” in a reference sequence) includes a set of one or more sequence positions indicated as capable of binding (“bindable”) to a molecule.

Based on knowledge of the positions constituting the strategic region, the engine may annotate the aligned reference sequences in the MSA 1402 with annotations indicating the one or more strategic regions (e.g., the fourth and tenth amino acid positions) to generate an annotated MSA (1410).

Referring to FIG. 8, the engine may then align a test sequence 1612 with the annotated reference alignment 1410 (1412), and add the aligned test sequence to the MSA 1410 to create a combined MSA 1413. The alignment may be performed using a multiple sequence alignment technique such as the MAFFT-add algorithm in the MAFFT software package. For example, MAFFT-add may perform the alignment based on comparing the amino acids at particular positions in the test sequence to those in the reference MSA. Here, amino acids E and V at positions 4 and 10, respectively, in the test sequence align with those positions in the reference MSA. Based on the alignment of the test sequence with the annotated MSA and knowledge that the strategic (reference) region includes the fourth and tenth positions, the engine may predict that position 4 and position 10 in the test sequence are strategic positions (target regions).

The question arises: If out of the 100,000 hits in the example of FIG. 7, the user wants to select ten test sequences for screening, how would that be accomplished? According to embodiments of the disclosure, the engine selects one or more test sequences as the one or more candidate sequences based at least in part upon the probability that a sequence component (e.g., an amino acid) occurs at a position in the one or more reference regions. For example, in FIG. 8, amino acid E has a probability p=1 at position 4 because in the five reference sequences Seq1-Seq5, E appears at position 4 in all five sequences. Under that logic, at position 10 amino acid V has p=0.6, amino acid L has p=0.2 (⅕), and amino acid I has p=0.2 (⅕).

In this example, amino acid E is universally conserved at position 4 in the binding region. This very likely indicates that E at that position may be necessary for a successful catalysis of a reaction involving diverse substrates. Thus, the engine may eliminate from consideration as a candidate sequence any test sequence that lacks amino acid E at position 4 of the binding region.

According to embodiments of the disclosure, to screen variations in amino acids at particular positions in the strategic region, the engine may account for the positional probabilities in the following manner. The prediction engine may select N (e.g., N=10) candidate sequences based on the probabilities of an amino acid occurring at a particular position. For example, if a user wants to screen ten sequences for variations in position 10 of Seq1 1602 of FIG. 8, the engine may select as candidates for screening six sequences with amino acid V at position 10 (based on p=0.6), two sequences with amino acid L at that position (based on p=⅕=0.2), and two positions with amino acid I at that position (based on p=⅕=0.2) (1414). A user may also want to screen ten sequences with variations at other positions (e.g., position 13 with 60% frequency of amino acid I) that, although it does not physically bind with the molecule, may support (e.g., stabilize) the molecule's binding to a strategic region. In that case, the engine may weight the selection of candidate sequences from the test set according to the probabilities of the amino acids at that position.

Based on the selection of candidate sequences according to either of the two general approaches above, the engine may instruct lab automation to react the candidate sequences (e.g., enzymes) with corresponding substrates and measure the resulting performance, e.g., yield or productivity of the desired reaction product of interest. Enzyme performance may be measured in terms of enzyme activity in units of moles of substrate converted per unit time, or in terms of enzyme processivity. In general, enzyme assays may measure either the consumption of substrate or production of product over time, usually by using four types of experiments: initial rate experiments; progress curve experiments; transient kinetics experiments; or relaxation experiments. Enzyme assays may employ two different types of sampling methods: continuous assays or discontinuous assays. An enzyme assay may also measure consumption of a cofactor (e.g., NADH) used by the enzyme.

Performance may be measured in other ways. For example, the performance of a transporter may be measured as the flux at which the transporter transports a molecule into a cell or across a membrane. The performance of a protein inhibitor may be measured based on the inactivation of a protein. For candidate sequences resulting in empirical performance that satisfy a threshold, the engine may add those sequences to the reference set for use as reference sequences in future iterations of the above algorithms.

According to embodiments of the disclosure, the candidate sequences identified by the two approaches discussed above may be combined for screening, e.g., some or all of the sequences identified in step 1414 may be combined with those identified in step 1464.

Machine Learning

Embodiments of the disclosure may apply machine learning (“ML”) techniques to learn the relationship between given parameters (sequences) and observed outcomes (e.g., functions). In this framework, embodiments may use standard ML models, e.g. Decision Trees, to determine feature importance. In general, machine learning may be described as the optimization of performance criteria, e.g., parameters, techniques or other features, in the performance of an informational task (such as classification or regression) using a limited number of examples of labeled data, and then performing the same task on unknown data. In supervised machine learning such as an approach employing linear regression, the machine (e.g., a computing device) learns, for example, by identifying patterns, categories, statistical relationships, or other attributes exhibited by training data. The result of the learning is then used to predict whether new data will exhibit the same patterns, categories, statistical relationships or other attributes.

Embodiments of this disclosure may employ unsupervised machine learning.

Alternatively, some embodiments may employ semi-supervised machine learning, using a small amount of labeled data and a large amount of unlabeled data. Embodiments may also employ feature selection to select the subset of the most relevant features to optimize performance of the machine learning model. Depending upon the type of machine learning approach selected, as alternatives or in addition to linear regression, embodiments may employ for example, logistic regression, neural networks, support vector machines (SVMs), decision trees, hidden Markov models, Bayesian networks, Gram Schmidt, reinforcement-based learning, cluster-based learning including hierarchical clustering, genetic algorithms, and any other suitable learning machines known in the art. In particular, embodiments may employ logistic regression to provide probabilities of classification along with the classifications themselves. See, e.g., Shevade, A simple and efficient algorithm for gene selection using sparse logistic regression, Bioinformatics, Vol. 19, No. 17 2003, pp. 2246-2253, Leng, et al., Classification using functional data analysis for temporal gene expression data, Bioinformatics, Vol. 22, No. 1, Oxford University Press (2006), pp. 68-76, all of which are incorporated by reference in their entirety herein.

Embodiments may employ graphics processing unit (GPU) or Tensor processing units (TPU) accelerated architectures that have found increasing popularity in performing machine learning tasks, particularly in the form known as deep neural networks (DNN). Embodiments of the disclosure may employ GPU-based machine learning, such as that described in GPU-Based Deep Learning Inference: A Performance and Power Analysis, NVidia Whitepaper, November 2015, Dahl, et al., Multi-task Neural Networks for QSAR Predictions, Dept. of Computer Science, Univ. of Toronto, June 2014 (arXiv:1406.1231 [stat.ML]), all of which are incorporated by reference in their entirety herein. Machine learning techniques applicable to embodiments of the disclosure may also be found in, among other references, Libbrecht, et al., Machine learning applications in genetics and genomics, Nature Reviews: Genetics, Vol. 16, June 2015, Kashyap, et al., Big Data Analytics in Bioinformatics: A Machine Learning Perspective, Journal of Latex Class Files, Vol. 13, No. 9, Sept. 2014, Prompramote, et al., Machine Learning in Bioinformatics, Chapter 5 of Bioinformatics Technologies, pp. 117-153, Springer Berlin Heidelberg 2005, all of which are incorporated by reference in their entirety herein.

Computer System Implementation

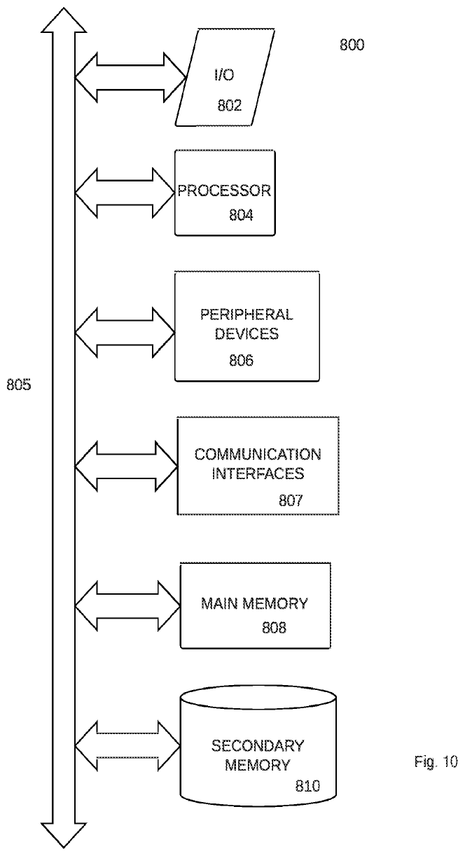

FIG. 9 illustrates a cloud computing environment 604 according to embodiments of the present disclosure. In embodiments of the disclosure, the software 610 for the engine may be implemented in a cloud computing system 602, e.g., to enable multiple users to implement embodiments of the present disclosure. Client computers 606, such as those illustrated in FIG. 10, access the system via a network 608, such as the Internet. The system may employ one or more computing systems using one or more processors, of the type illustrated in FIG. 10. The cloud computing system itself includes a network interface 612 to interface application software 610 (for performing operations of embodiments of the disclosure) to the client computers 606 via the network 608. The network interface 612 may include an application programming interface (API) to enable client applications at the client computers 606 to access the system software 610. In particular, through the API, client computers 606 may access the engine.

A software as a service (SaaS) software module 614 offers the system software 610 as a service to the client computers 606. A cloud management module 616 manages access to the system 610 by the client computers 606. The cloud management module 616 may enable a cloud architecture that employs multitenant applications, virtualization or other architectures known in the art to serve multiple users.

FIG. 10 illustrates an example of a computer system 800 that may be used to execute program code stored in a non-transitory computer readable medium (e.g., memory) in accordance with embodiments of the disclosure. The computer system includes an input/output subsystem 802, which may be used to interface with human users or other computer systems depending upon the application. The I/O subsystem 802 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the engine, may be implemented with a computer system like that of computer system 800.

Program code may be stored in non-transitory media such as persistent storage in secondary memory 810 or main memory 808 or both. Main memory 808 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 804 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 804. The processor(s) 804 may include graphics processing units (GPUs) for handling computationally intensive tasks.

The processor(s) 804 may communicate with external networks via one or more communications interfaces 807, such as a network interface card, WiFi transceiver, etc. A bus 805 communicatively couples the I/O subsystem 802, the processor(s) 804, peripheral devices 806, communications interfaces 807, memory 808, and persistent storage 810. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 800. In particular, the engine and any other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 9.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references cited herein, including, without limitation, articles, publications, patents, patent publications, and patent applications, are incorporated by reference in their entireties for all purposes, except that any portion of any such reference is not incorporated by reference herein if it: (1) is inconsistent with embodiments of the disclosure expressly described herein; (2) limits the scope of any embodiments described herein; or (3) limits the scope of any terms of any claims recited herein. Mention of any reference, article, publication, patent, patent publication, or patent application cited herein is not, and should not be taken as an acknowledgment or any form of suggestion that it constitutes valid prior art or forms part of the common general knowledge in any country in the world, or that it discloses essential matter.

In the claims below, a claim n reciting “any one of the preceding claims starting with claim x,” shall refer to any one of the claims starting with claim x and ending with the immediately preceding claim (claim n−1). For example, claim 35 reciting “The system of any one of the preceding claims starting with claim 28” refers to the system of any one of claims 28-34.

1. A method for identifying candidate biological sequences for screening to determine whether the candidate biological sequences enable a biological function, the method comprising:
identifying, by one or more processors, a plurality of candidate sequences in a test set of test sequences based at least in part upon
(a) degrees of similarity between the test sequences and reference sequences, of a reference set of reference sequences, that are known to enable the function, and
(b) comparisons of one or more regions in the test sequences to one or more reference regions, in one or more of the reference sequences, that are known to bind to a molecule.

2. The method of claim 1, wherein identifying comprises:
determining a set of matching test sequences including test sequences having degrees of sequence similarity to the reference sequences that satisfy a similarity threshold; and
comparing one or more regions of the matching test sequences with the one or more reference regions.

3. The method of claim 1, wherein identifying a plurality of candidate sequences comprises employing a Hidden Markov Model (“HMM”) to determine the degrees of similarity between the test sequences and the reference sequences.

4. The method of claim 1, wherein the one or more reference regions are identified based at least in part upon an analysis of three-dimensional structures of the one or more reference sequences.

5. The method of claim 1, wherein a multiple sequence alignment (“MSA”) of the reference sequences is annotated with annotations indicating the one or more reference regions, and the test sequences are added to the annotated reference MSA after aligning the test sequence to the MSA.

6. The method of claim 1, wherein the comparisons of the one or more regions in the test sequences to the one or more reference regions comprise selecting a plurality of test sequences as the plurality of candidate sequences based at least in part upon the probability that a sequence component occurs at a position in the one or more reference regions.

7. The method of claim 1, wherein empirical performance of one or more selected candidate sequences is determined.

8. The method of claim 1, further comprising adding one or more first candidate sequences to the reference set based at least in part upon empirical performance of the one or more first candidate sequences.

9. A method for producing a desired molecule, the method comprising manufacturing a desired molecule employing one or more candidate biological sequences that enable one or more functions used to produce the desired molecule, wherein the one or more candidate biological sequences that enable one or more functions used to produce the desired molecule are identified using the method of claim 1.

10. The method of claim 9, wherein the one or more candidate biological sequences are enzymes that catalyze at least one reaction in a pathway leading to the desired molecule.

11. (canceled)

12. (canceled)

13. A method for identifying, in a test set of test sequences, candidate biological sequences for screening to determine whether they enable a biological function, the method comprising:
a. determining sequence similarities between reference sequences, of a reference set of reference sequences, that are known to enable the biological function;
b. grouping the reference sequences into clusters based at least in part upon their sequence similarities, wherein each cluster corresponds to a molecule that is indicated as bindable by one or more of the reference sequences in the cluster; and
c. identifying a plurality of matching test sequences based upon a comparison of the test sequences with reference sequences in one or more of the clusters.

14. The method of claim 13, comprising employing a Hidden Markov Model (“HMM”) to determine the similarities between the test sequences and the reference sequences in the one or more clusters.

15. The method of claim 13, wherein the sequence similarities between the reference sequences are statistical estimates.

16. The method of claim 13, further comprising: identifying matching test sequences based upon the comparison of the test sequences with reference sequences in one or more of the clusters; clustering the matching test sequences into a plurality of clusters of the matching test sequences;
and selecting the candidate sequences from the plurality of clusters of the matching test sequences.

17. The method of claim 13, wherein empirical performance of one or more selected candidate sequences is determined.

18. The method of claim 13, further comprising adding one or more first candidate sequences to the reference set based at least in part upon empirical performance of the one or more first candidate sequences.

19. A method for producing a desired molecule, the method comprising manufacturing a desired molecule employing one or more candidate biological sequences that enable one or more functions used to produce the desired molecule, wherein the one or more candidate biological sequences that enable one or more functions used to produce the desired molecule are identified using the method of claim 13.

20. The method of claim 19, wherein the one or more candidate biological sequences are enzymes that catalyze at least one reaction in a pathway leading to the desired molecule.

21. (canceled)

22. (canceled)

23. A method for screening candidate biological sequences, the method comprising screening the candidate biological sequences to determine whether they enable a biological function, wherein the candidate biological sequences are identified by the method of claim 1.

24. (canceled)

25. One or more non-transitory computer-readable media storing instructions that, when executed by one or more computing devices, cause at least one of the one or more processors to screen candidate biological sequences, of a test set of test sequences, to determine whether they enable a biological function, wherein the candidate biological sequences have been identified performance of based at least in part upon
(a) degrees of similarity between the test sequences and reference sequences, of a reference set of reference sequences, that are known to enable the function, and
(b) comparisons of one or more regions in the test sequences to one or more reference regions, in one or more of the reference sequences, that are known to bind to a molecule.

26. A method for screening candidate biological sequences, the method comprising screening the candidate biological sequences to determine whether they enable a biological function, wherein the candidate biological sequences are identified by the method of claim 13.


SEQUENCE LISTING (10 sequences)

SEQ ID NO: 1 - protein, 497 aa, Oryza sativa
MEGVGGGGGGEEWLRPMDAEQLRECGHRMVDFVADYYKSIEAFPVLSQVQPGYLKEVLPDSAPRQPDTLDSLFDDIQQKIIPGVTHWQSPNYFAYYPSNSSTAGFLGEMLSAAFNIVGFSWITSPAATELEVIVLDWFAKMLQLPSQFLSTALGGGVIQGTASEAVLVALLAARDRALKKHGKHSLEKLVVYASDQTHSALQKACQIAGIFSENVRVVIADCNKNYAVAPEAVSEALSIDLSSGLIPFFICATVGTTSSSAVDPLPELGQIAKSNDMWFHIDAAYAGSACICPEYRHHLNGVEEADSFNMNAHKWFLTNFDCSLLWVKDRSFLIQSLSTNPEFLKNKASQANSVVDFKDWQIPLGRRFRSLKLWMVLRLYGVDNLQSYIRKHIHLAEHFEQLLLSDSRFEVVTPRTFSLVCFRLVPPTSDHENGRKLNYDMMDGVNSSGKIFLSHTVLSGKFVLRFAVGAPLTEERHVDAAWKLLRDEATKVLGKMV

SEQ ID NO: 2 - protein, 575 aa, Modestobacter marinus
MTGHMTPEQFRQHGHEVVDWIADYWERIGSFPVRSQVSPGDVRASLPPTAPEQGEPFSAVLADLDRVVLPGVTHWQHPGFFGYFPANTSGPSVLGDLVSAGLGVQGMSWVTSPAATELEQHVMDWFADLLGLPESFRSTGSGGGVVQDSSSGANLVALLAALHRASKGATLRHGVRPEDHTVYVSAETHSSMEKAARIAGLGTDAIRIVEVGPDLAMNPRALAQRLERDVARGYTPVLVCATVGTTSTTAIDPLAELGPICQQHGVWLHVDAAYAGVSAVAPELRALQAGVEWADSYTTDAHKWLLTGFDATLFWVADRAALTGALSILPEYLRNAATDTGAVVDYRDWQIELGRRFRALKLWFVVRWYGAEGLREHVRSHVALAQELAGWADADERFDVAAPHPFSLVCLRPRWAPGIDADVATMTLLDRLNDGGEVFLTHTTVDGAAVLRVAIGAPATTREHVERVWALLGEAHDWLARDFEEQAAERRAAELREREAAEEQLRARREAEAAAAAATEAPVEPAAEEPEQLVVPPVEVPAVETPAAWDESATQVAAQTDLHADPAPQPADGQG

SEQ ID NO: 3 - protein, 481 aa, Streptomyces sviceus
MPDLEPDEFRRQGHQLVDWVARYRTSLPSLHVRPKVVPGSVKAQLPRELPEQPSQALGDDLIALLNDVVVPSSLHWQHPGFFGYFPANASLLSLLGDIASGGIGAQGMLWSTSPAGTEIEQVLLDGLADALGLGREFTFAGGGGGSLQDSASSASLAALLAALQRSNPDWREHGVDGTETVYVTAETHSSLAKAVRVAGLGARALRIVPFTQGTLSMSADALADMLAKDTAAGKRPVMVCPTVGTTGTGAIDPVREVALAARTYEAWVHVDAAWAGVAALCPEFRWLLDGVNLVDSFCTDAHKWFYTAFDASFMWVRDARALPTALSITPEYLRNAATESGEVIDYRDWQVPLGRRMRALKIWSVVHGAGLEGLRESIRGHVAMANSLAGRIESESGFALATPPSLALVCLYLVDQEGRPDDAATKAAMEAVNAEGHSFLTHTSVNGHFAIRVAIGATTTLPDHIDTLWDSLCKAARQSGG

SEQ ID NO: 4 - protein, 470 aa, Pseudomonas putida
MTPEQFRQYGHQLIDLIADYRQTVGERPVMAQVEPGYLKAALPATAPQQGEPFAAILDDVNNLVMPGLSHWQHPDFYGYFPSNGTLSSVLGDFLSTGLGVLGLSWQSSPALSELEETTLDWLRQLLGLSGQWSGVIQDTASTSTLVALISARERATDYALVRGGLQAEPKPLIVYVSAHAHSSVDKAALLAGFGRDNIRLIPTDERYALRPEALQAAIEQDLAAGNQPCAVVATTGTTTTTALDPLRPVGEIAQANGLWLHVDSAMAGSAMILPECRWMWDGIELADSVVVNAHKWLGVAFDCSIYYVRDPQHLIRVMSTNPSYLQSAVDGEVKNLRDWGIPLGRRFRALKLWFMLRSEGVDALQARLRRDLDNAQWLAGQVEAAAEWEVLAPVQLQTLCIRHRPAGLEGEALDAHTKGWAERLNASGAAYVTPATLDGRWMVRVSIGALPTERGDVQRLWARLQDVIKG

SEQ ID NO: 5 - protein, 384 aa, Propionibacterium sp.
MGMDISSRPVEWASLSEITASDVSFEGGAIFNSICTRPHPLAAQVMADNLHLNAGDGRLFPSVARCESEITNFLGGLMGLPRAVGMCTSGATEANLIAVHSAIENWRRKGGQGRPQVILGRGGHFSFDKISVLLGVELVLAWSDIDTLKVDPESVSELISPRTALIVATAGSSETGAVDDVEWLSRVALSKGVPLHVDAASGGLLIPFLRDLGGALPDIGFRNDGVTTIAIDPHKFGSAPIPSGHLVAREWTWIEGLRTESHYQGTARHLTFLGTRSGGSILATYALFGHLGEKGLRGMAEQLKALRSHLVDRLRKAGATLAYVPELMVVALKADSDAVKVLERRGIFTSYAKRLGYLRIVVQLHMSEGQVDGLVDALLMEGIV

SEQ ID NO: 6 - protein, 361 aa, Enterococcus faecium
TKLQNNELKRGWGHIVADGSLANLEGLWYARNIKSLPLAMKEVTPELVAGKSDWELMNLSTEEIMNLLDSVPEKIDEIKAHSARSGKHLEKLGKWLVPQTKHYSWLKAADIIGIGLDQVIPVPVDHNYRMDINELEKIVRGLAAEKTPILGVVGVVGSTEEGAIDGIDKIVALRRVLEKDGIYFYLHVDAAYGGYGRAIFLDEDNNFIPFEDLKDVHYKYNVFTENKDYILEEVHSAYKAIEEAESVTIDPHKMGYVPYSAGGIVIKDIRMRDVISYFATYVFEKGADIPALLGAYILEGSKAGATAASVWAAHHVLPLNVTGYGKLMGASIEGAHRFYNFLKDLSFKVGTKNRSSSITTH

SEQ ID NO: 7 - protein, 363 aa, Methanosphaerula palustris
MLNKGLAEEELFSFLSKKREEDLCHSHILSSMCTVPHPIAVKAHLMFMETNLGDPGLFPGTASLERLLIERLGDLFHHREAGGYATSGGTESNIQALRIAKAQKKVDKPNVVIPETSHFSFKKACDILGIQMKTVPADRSMRTDISEVSDAIDKNTIALVGIAGSTEYGMVDDIGALATIAEEEDLYLHVDAAFGGLVIPFLPNPPAFDFALPGVSSIAVDPHKMGMSTLPAGALLVREPQMLGLLNIDTPYLTVKQEYTLAGTRPGASVAGALAVLDYMGRDGMEAVVAGCMKNTSRLIRGMETLGFPRAVTPDVNVATFITNHPAPKNWVVSQTRRGHMRIICMPHVTADMIEQFLIDIGE

SEQ ID NO: 8 - protein, 432 aa, Petroselinum crispum
EFRRQGHLMIDFLADYYRKVENYPVRSQVSPGYLREILPESAPYNPESLETILQDVQTKIIPGITHWQSPNFFAYFPSSGSTAGFLGEMLSTGFNVVGFNWMVSPAATELENVVTDWFGKMLQLPKSFLFSGGGGGVLQGTTCEAILCTLVAARDKNLRQHGMDNIGKLVVYCSDQTHSALQKAAKIAGIDPKNFRAIETSKSSNFKLCPKRLESAILYDLQNGLIPLYLCATVGTTSSTTVDPLPALTEVAKKYKLWVHVDAAYAGSACICPEFRQYLDGVENADSFSLNAHKWFLTTLDCCCLWVRDPSALIKSLSTYPEFLKNNASETNKVVDYKDWQIMLSRRFRALKLWFVLRSYGVGQLREFIRGHVGMAKYFEGLVGMDNRFEVVAPRLFSMVCFRIKPSAMIGKNDEDEVNEINRKLLESVNDS

SEQ ID NO: 9 - protein, 396 aa, Methanocaldococcus jannaschii
MRNMQEKGVSEKEILEELKKYRSLDLKYEDGNIFGSMCSNVLPITRKIVDIFLETNLGDPGLFKGTKLLEEKAVALLGSLLNNKDAYGHIVSGGTEANLMALRCIKNIWREKRRKGLSKNEHPKIIVPITAHFSFEKGREMMDLEYIYAPIKEDYTIDEKFVKDAVEDYDVDGIIGIAGTTELGTIDNIEELSKIAKENNIYIHVDAAFGGLVIPFLDDKYKKKGVNYKFDFSLGVDSITIDPHKMGHCPIPSGGILFKDIGYKRYLDVDAPYLTETRQATILGTRVGFGGACTYAVLRYLGREGQRKIVNECMENTLYLYKKLKENNFKPVIEPILNIVAIEDEDYKEVCKKLRDRGIYVSVCNCVKALRIVVMPHIKREHIDNFIEILNSIKRD

SEQ ID NO: 10 - protein, 531 aa, Papaver somniferum
MGSLNTEDVLENSSAFGVTNPLDPEEFRRQGHMIIDFLADYYRDVEKYPVRSQVEPGYLRKRLPETAPYNPESIETILQDVTTEIIPGLTHWQSPNYYAYFPSSGSVAGFLGEMLSTGFNVVGFNWMSSPAATELESVVMDWFGKMLNLPESFLFSGSGGGVLQGTSCEAILCTLTAARDRKLNKIGREHIGRLVVYGSDQTHCALQKAAQVAGINPKNFRAIKTFKENSFGLSAATLREVILEDIEAGLIPLFVCPTVGTTSSTAVDPISPICEVAKEYEMWVHVDAAYAGSACICPEFRHFIDGVEEADSFSLNAHKWFFTTLDCCCLWVKDPSALVKALSTNPEYLRNKATESRQVVDYKDWQIALSRRFRSLKLWMVLRSYGVTNLRNFLRSHVKMAKTFEGLICMDGRFEITVPRTFAMVCFRLLPPKTIKVYDNGVHQNGNGVVPLRDENENLVLANKLNQVYLETVNATGSVYMTHAVVGGVYMIRFAVGSTLTEERHVIYAWKILQEHADLILGKFSEADFSS